(19) United States
(12) Patent Application Publication
(10) Pub. No.: US 2004/0024068 A1
(43) Pub. Date: Feb. 5, 2004

(54) Antimicrobial compounds

(75) Inventors: Stuart B. Levy, Boston, MA (US); Laura M. McMurry, Somerville, MA (US)

Correspondence Address: LAHIVE & COCKFIELD, 28 STATE STREET, BOSTON, MA, 02109, US

(73) Assignee: Trustees of Tufts College

(21) Appl. No.: 10/377,250

(22) Filed: Feb. 27, 2003

Related U.S. Application Data
(63) Continuation of application No. 09/235,896, filed on Jan. 22, 1999, now abandoned.
Application No. 09/235,896 is a continuation-in-part of application No. 09/013,440, filed on Jan. 26, 1998, now abandoned.
(60) Provisional application No. 60/072,244, filed on Jan. 23, 1998.

(51) Int. Cl. A61K 31/19; G01N 33/554; G01N 33/569

(52) U.S. Cl. 514/575; 435/7.32

(57) ABSTRACT

Methods and mutants for identifying an antimicrobial compound which interacts with an ER polypeptide are disclosed. In particular, the method pertains to screens for identifying an antimicrobial compound using FabI or InhA mutant cells or polypeptides.

### RELATED APPLICATION

This application claims priority to U.S. provisional application Serial No. 60/072,244 filed Jan. 23, 1998, and to U.S. application Ser. No. 09/013,440, filed on Jan. 26, 1998, the contents of which are entirely incorporated by reference, along with the attached Appendices.

### BACKGROUND OF THE INVENTION

Triclosan is a trichlorinated biphenyl broad spectrum antibacterial/fungal agent (Furia, T. E., et al. Soap & Chemical Specialties 44, 47-50, 116-122 (1968); Regos, J., et al. Dermatologica 158, 72-79 (1979)). Because of its general biocidal activity, triclosan has been used as a topical disinfectant in soaps, cosmetics, and lotions (Regos, J., et al. Dermatologica 158, 72-79 (1979)), and more recently has been added to toothpastes (Cummins, D. J. Clin. Periodont. 18, 455-461 (1991), to fabrics for use in bedding and clothing, and to plastics for use in toys, cutting boards, and flooring.

The mechanism of action of triclosan has been uncertain; biochemical and physical assays have shown inhibition of uptake of nutrients (Regos, J., et al. Zbl. Bakt. Hyg., I. Abt. Orig A 226, 390-401 (1974)), inhibition of proteases (Cummins, D. J. Clin. Periodont. 18, 455-461 (1991)), and cell lysis (Regos, J., et al. Zbl. Bakt. Hyg., I. Abt. Orig. A 226, 390-401 (1974)); Cummins, D. J. Clin. Periodont. 18, 455-461 (1991)). A plasmid-mediated triclosan resistance has been reported in Staphylococcus aureus but the mechanism is unknown. (Cookson, B. D., et al. The Lancet 337, 1548-1549 (1991)).

### SUMMARY OF THE INVENTION

The present invention is based, at least in part, on the discovery that triclosan (an antimicrobial compound commonly used in consumer products, e.g., soaps and detergents), has a genomic target which is involved in its ability to impart antimicrobial activity. The present invention further includes the identification of the genomic target for triclosan in Escherichia coli and in Mycobacterium smegmatis, as FabI and InhA, respectively, and provides for methods of identifying antimicrobial compounds based upon this identification (hereinafter screening assays will be used interchangeably for such methods for discussion purposes).

The present invention also is based, at least in part, on the discovery of triclosan-resistant microbial cells and the identification of mutant enoyl ACP-reductase (ER) polypeptides, e.g., E. coli FabI polypeptides or M. smegmatis InhA polypeptides, which confer the triclosan-resistance to the microbial cells. (For discussion purposes below, the term ER will be used to refer to these reductase enzymes, and it should be understood that the descriptions apply to the ER polypeptide as well as to the FabI and InhA polypeptide embodiments.) The present invention includes the development of screening assays using these mutant polypeptides and triclosan-resistant microbial cells for antimicrobial compounds which can be used against triclosan-resistant microbial cells, e.g., in lieu of triclosan or in addition to triclosan.

It should be appreciated that the present invention is the first time that a non-specific antimicrobial agent (hereinafter NSAM) was shown to be target specific on a genomic level, i.e., have a genomic target which is involved in its ability to impart antimicrobial activity. NSAMs for the purpose of this invention is intended to include the broad class of antimicrobial compounds, e.g., found in consumer products, that (prior to the present discovery) were not believed to be target specific on the genomic level by those of ordinary skill in the art. NSAMs are not intended to include antibiotics or other antimicrobials which one of ordinary skill in the art would have expected to be target specific on a genomic level prior to the discovery of the present invention. The present invention includes the identification of genomic targets involved in an NSAM's ability to impart antimicrobial activity and the development of screening assays for antimicrobial compounds based upon these genomic targets.

The invention features identification of a second genomic target that influences cell sensitivity to triclosan, the efflux pump which is the product of the acrAB gene. In one embodiment, the invention describes double mutants altered both in er and acrAB, such that inactivation of the efflux pump renders both er⁺ (wild type) and er mutant cells more sensitive to triclosan. This embodiment provides that, in the presence of an inhibitor of the AcrAB efflux pump, a lower effective dose of an inhibitor of an ER protein is required to effectively inhibit the ER protein and achieve biocidal, antimicrobial, or antibiotic activity.

Other aspects of the invention include the reagents used in the aforementioned screening assays, antimicrobial compounds identified using the screening assays, and methods of using the identified compounds in combination products, e.g., consumer products and in therapeutic methods.

### BRIEF DESCRIPTION OF THE DRAWING

FIG. 1 is a schematic of a restriction map of the pLYT8 region encoding triclosan resistance, and deletion mutants. The thick (gray or black) horizontal region represents chromosomal DNA inserted into the tet gene of the pBR322 vector (thin, white). The deleted regions of the mutants are represented by interruptions of the black horizontal line; pLYT11 was created using BsmI and pLYT12 using SspI. The response to triclosan (MIC, μg ml⁻¹) encoded by the plasmids in hypersusceptible host strain AG100A are the numbers shown in parentheses.

FIG. 2 is a diagram illustrating an exemplary alignment of the protein sequences of Fab1 and InhA.

### DETAILED DESCRIPTION OF THE INVENTION

The present invention pertains to a method for identifying an antimicrobial compound which interacts with an ER polypeptide, e.g., a FabI or InhA polypeptide. (For discussion purposes below, the term ER will be understood to the ER polypeptide and to the FabI and InhA polypeptide embodiments.) The method involves contacting the ER polypeptide with a compound under conditions which allow interaction of the compound with the ER polypeptide to occur. The method further includes detecting the interaction of the compound with the ER polypeptide as an indication of whether the compound is an antimicrobial compound.

The language “antimicrobial compound” is art-recognized and is intended to include a compound which inhibits the proliferation or viability of a microbe which is undesirable and/or which disrupts a microbial cell. The language further includes significant diminishment of a biological activity which is undesirable and associated with the microbe, such that a subject would not be detrimentally affected by the microbe. Examples include antibiotics, biocides, antibacterial compounds.

The language “ER polypeptide” is intended to include a polypeptides having enoyl-acyl carrier protein reductase activity. The ER polypeptides of the present invention include full length ER polypeptides and/or biologically active fragments thereof. The preferred fragments contain the reducing agent binding cleft and/or the triclosan binding portion and/or the substrate binding site, and are of a size which allows for their use in the screening methods of the present invention. An example of such a polypeptide is a FabI or InhA polypeptide. In addition to these two exemplary ER polypeptides, the term ER polypeptide is also meant to cover ER polypeptides from other microorganisms, e.g., from species other than E. coli or M. smegmatis.

The language “Fab1” and “InhA” is art-recognized and is intended to exemplify ER polypeptides having enoyl-acyl carrier protein reductase activity. The Fab1 and InhA polypeptides of the present invention include the full length polypeptides and/or biologically active fragments thereof. The preferred fragments contain the reducing agent binding cleft and/or the triclosan binding portion and/or the substrate binding site, and are of a size which allows for their use in the screening methods of the present invention. ER polypeptides of the present invention are discussed in further detail below.

The term “compound” is art-recognized and includes compounds being tested for antimicrobial activity. The compound can be designed to incorporate a moiety known to interact with a ER polypeptide or can be selected from a library of diverse compounds, e.g., based on a desired activity, e.g., random drug screening based on a desired activity. Preferably, the compound of the present invention is a small molecule. Examples of compounds of the present invention include NSAMs and triclosan compounds.

“NSAM” for the purpose of this invention is as defined above. An NSAM compound includes functional and structural analogs of a parent NSAM compound. The analogs can be selected or designed either using the genomic target involved in its ability to impart antimicrobial activity and/or based upon knowledge derived from studying the interaction between the NSAM and the genomic target.

The language “triclosan compound” includes functional and structural analogs of triclosan. The analogs can be selected or designed either using the genomic target and/ or based upon knowledge derived from studying the interaction between triclosan and the genomic target.

In one embodiment the compound is not an antibiotic. In another embodiment, the compound is not isoniazid, diazaborine, or ethionamide.

The compound can be a single compound or can be a member of a test library. Exemplary test libraries that can be used include combinatorial libraries or libraries of natural products.

The synthesis of combinatorial libraries is well known in the art and has been reviewed (see, e.g., E. M. Gordon et al., J. Med. Chem. (1994) 37:1385-1401; DeWitt, S. H.; Czamik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

The language “interacts with an ER polypeptide” include interactions with the polypeptide which result in the identification of a compound having antimicrobial activity. Such interactions include binding of the compound to the polypeptide, e.g., direct or indirect binding, which allows for identification of a compound having antimicrobial activity. In one embodiment, the interaction occurs with the reducing agent binding cleft of the ER polypeptide. In another embodiment, the interaction occurs with the triclosan binding portion of the ER polypeptide.

The language “reducing agent binding cleft” is intended to include that portion of the ER polypeptide which interacts with, e.g., binds with, a reducing agent. An example of a reducing agent cleft is the NAD (or NADH⁺)/NADP (or NADPH⁺) binding cleft of the ER polypeptide.

The language “triclosan binding portion” is that portion of the ER polypeptide which binds, e.g. directly or indirectly, triclosan. In one embodiment the triclosan binding portion is within the reducing agent binding cleft.

The language “detecting the interaction of the compound with the ER polypeptide” includes means of detection which result in the identification of a compound having antimicrobial activity. For example, the interaction can be detected based on the presence or absence of enzyme activity, e.g., using art-recognized techniques.

The present invention further pertains to a method for identifying an antimicrobial compound by contacting an enoyl reductase molecule with a compound under conditions which allows enzyme activity to occur. In this method, the presence or absence of enzyme activity is detected as an indication of whether the compound is an antimicrobial compound.

The language and terms of this method are as defined above and/or below. The language “enoyl reductase molecule” (ER) is art recognized and is a cytoplasmic enzyme involved in the synthesis of fatty acids. The enzymatic activity can be measured using art-recognized techniques some of which are discussed below.

The present invention further pertains to a method for identifying an antimicrobial compound by exposing or contacting a microorganism to a compound under conditions which allow fatty acid biosynthesis to occur. In this method, the inhibition of fatty acid biosynthesis is detected as an indication of whether the compound is an antimicrobial compound. The language and terms of this method are as defined above and/or below.

The language “inhibition of fatty acid biosynthesis” is art recognized and includes the inhibition of the synthesis of at least one fatty acid in the microorganism. The inhibition of fatty acid biosynthesis can be measured as discussed below. The term “microorganism” is art- recognized and for purposes of this invention is used interchangeably with “microbe or microbial cell”.

The present invention further pertains to a method for identifying an antimicrobial compound which interacts with a mutant ER polypeptide by contacting the mutant ER polypeptide with a compound under conditions which allow interaction of the compound with the mutant ER polypeptide to occur. In this method, the presence or absence of interaction of the compound with the mutant ER polypeptide is detected as an indication of whether the compound is an antimicrobial compound. The language and terms of this method are as defined above and below.

The language “mutant of an ER polypeptide” is intended to include polypeptides which differ from the ER polypeptide in an alteration of at least one amino acid residue but retain their ability to be useful within the screening assays of the present invention. The mutant ER polypeptides of the present invention include the full length mutant ER polypeptide and/or biologically active fragments thereof. The preferred fragments contain the reducing agent binding cleft and/ or the triclosan binding portion and are of a size which allows for their use in the screening methods of the present invention.

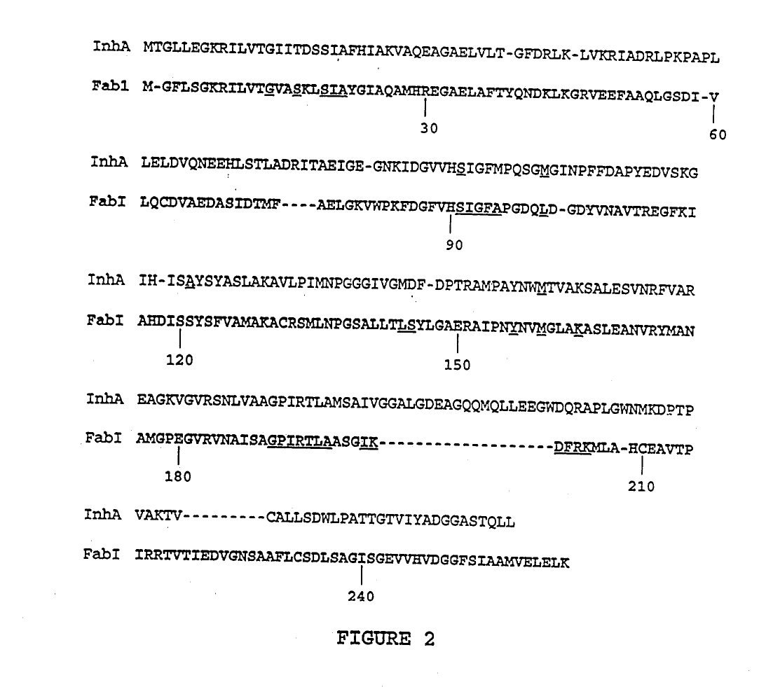

In one embodiment, the protein product of the mutant gene is capable of conferring resistance to triclosan in a microbial cell. In another embodiment, the protein product of the mutant gene is capable of conferring resistance to an NSAM in a microbial cell. In another embodiment, the mutant has a gly93val substitution. (The convention used here to describe the substitution mutation lists the wild-type amino acid followed by the position of the residue in the protein followed by the substituted mutant amino acid.) In another embodiment, the mutant has a met159thr or phe203leu substitution. In another embodiment, the mutant has an alteration of at least one amino acid in the reducing agent, e.g., NAD/NADP binding cleft of the ER molecule or an alteration of at least one amino acid residue in the triclosan binding portion. In even more specific embodiments the mutant ER protein is a mutant Fab1 polypeptide having an amino acid sequence as shown in SEQ ID NO: 3 except that it comprises an amino acid substitution at a position selected from the group consisting of G13, S16, S19, I20, A21, S91, I92, G93, F94, A95, L100, L144, S145, Y156, M159, K163, G190, P191, I192, R193, T194, L195, A196, 1200, K201, D202, F203, R204 and K205. Exemplary residues for substitution underlined in FIG. 2. One of ordinary skill in the art would understand that the numbering system is based on the E. coli FabI polypeptide. Based on this finding, one of ordinary skill in the art would further be able to select comparable residues which are applicable to another microorganism. For example, an alignment of FabI can be made with other, related ER molecules. An exemplary alignment of FabI and InhA, made using the BLAST algorithm, is shown in FIG. 2. Using such an alignment, it is possible to determine mutations in other ER polypeptides that would correspond to mutations in a FabI or InhA polypeptide which have been shown to confer resistance to triclosan. As used herein, the language “corresponds to” is meant to include an approximate correspondence when the sequence are aligned in a biologically meaningful manner by one of ordinary skill in the art. The language “corresponds to” also includes residues which spatially correspond, e.g., are in the same functional position upon crystallography, but which may not correspond when aligned using an alignment program. The language “corresponds to” also includes residues which perform the same function, e.g., mediate an enzymatic activity or bind the same cofactor.

Other exemplary mutant ER proteins include, e.g., InhA mutants Ser94Ala (corresponding to FabI S91); Met103Thr (corresponding to FabI L100); Ala124Val (corresponding to FabI S121); Met161Val (corresponding to FabI M159). Mutant ER polypeptides are discussed in further detail below.

The present invention further pertains to a method for identifying an antimicrobial compound capable of inhibiting proliferation or viability of a triclosan-resistant microbial cell. The method involves contacting a triclosan-resistant microbial cell with a compound under conditions which allow a triclosan-resistant microbial cell to proliferate or remain viable. The method further includes determining whether the compound is capable of inhibiting proliferation or viability of the cell thereby identifying an antimicrobial compound capable of inhibiting proliferation or viability of a triclosan-resistant microbial cell. The language and terms of this method are as defined above and/or below.

The language “triclosan-resistant microbial cell” is intended to include a microbial cell which has become resistant to the antimicrobial effect(s) of triclosan, e.g., triclosan no longer inhibits the proliferation of the microbial cell or the cell remains viable when exposed to triclosan, at a concentration of triclosan sufficient to kill the parent sensitive cell. Sensitivity is measured by a parameter known as “minimum inhibitory concentration” (MIC), such that a triclosan-resistant microbial cell has a MIC that is at least 1.5-fold greater than the sensitive parent, at least 2-fold greater than the sensitive parent, preferably at least 4-fold greater than the sensitive parent, even more preferably at least 10-fold greater than the sensitive parent. Examples of triclosan-resistant microbial cell include the cell lines described in the examples below such as AGT11, AGT23, and AGT25. The triclosan-resistant microbial cell also can be acrAB⁺, i.e., it possesses at least the efflux pump protein of the acrAB⁺ gene, such that triclosan sensitivity is enhanced by genetic loss of this gene, or by chemical inhibition of its activity.

The inhibition of proliferation or viability of the cell can be determined or can be detected using art-recognized techniques, e.g., optical detection. For example, the presence of lysis of the triclosan-resistant microbial cell can be used to identify an antimicrobial compound capable of inhibiting proliferation or viability, and/or disrupting, a triclosan-resistant microbial cell.

The present invention further pertains to a method for identifying an antimicrobial compound capable of inhibiting proliferation or viability of a triclosan-resistant microbial cell by contacting a polypeptide capable of conferring resistance to triclosan with a compound under conditions which allow interaction of the compound to the polypeptide to occur. In this method, the presence or absence of interaction of the compound with the polypeptide is detected as an indication of whether the compound is an antimicrobial compound capable of inhibiting proliferation or viability of a triclosan-resistant microbial cell. The language and terms of this method are as defined above and/or below.

The language “polypeptide capable of conferring resistance to triclosan” is intended to include a polypeptide which when present in the microbial cell under appropriate conditions confers resistance to triclosan to the microbial cell, e.g., the microbial cell can proliferate and remain viable in the presence of triclosan.

The invention further pertains to a method for identifying an antimicrobial compound capable of inhibiting proliferation or viability of a NSAM-resistant microbial cell. The method involves contacting a polypeptide capable of conferring resistance to a NSAM with a compound under conditions which allow interaction of the compound with the polypeptide to occur. The method further involves detecting the presence or absence of interaction with the polypeptide as an indication of whether the compound is an antimicrobial compound capable of inhibiting proliferation or viability of a NSAM-resistant microbial cell. The language and terms of this method are as defined above and/or below.

The language “polypeptide capable of conferring resistance to a NSAM” is intended to include a polypeptide which when present in the microbial cell under appropriate conditions confers resistance to a NSAM to the microbial cell, e.g., the microbial cell can proliferate and remain viable in the presence of the NSAM.

Other aspects of this invention include antimicrobial compounds identified using any of the aforementioned methods or screening assays and the use of these compounds in combination products or in therapy as an active agent in a pharmaceutical composition.

The “combination product” includes an antimicrobial compound identified using a screening method of the invention and a product forming the combination product. The term “product” is intended to include consumer products such as detergents, soaps, deodorant mouthwash, toothpaste, and lotions.

The present invention further pertains to a combination product containing a structural analog of triclosan and a product forming a combination product. In a preferred embodiment, the combination product containing the structural analog of triclosan is effective against a triclosan-resistant microbial cell.

The present invention further pertains to a combination product containing a structural analog of an NSAM and a product forming a combination product. In a preferred embodiment, the combination product containing the structural analog of the NSAM is effective against a triclosan-resistant microbial cell or an NSAM-resistant microbial cell.

The present invention further pertains to a method for inhibiting the growth of an unwanted microorganism with a NSAM or an NSAM compound by administering to the subject an effective amount of the NSAM or the NSAM compound such that the growth of the unwanted microorganism is inhibited. The present invention even further pertains to a method for inhibiting the growth of an unwanted microorganism with a triclosan compound or with the parent triclosan compound by contacting a surface, e.g., the surface of an instrument, the surface of the skin of a subject, the surface of a room, or the surface of a container, with an effective amount of the NSAM such that the growth of the unwanted microorganism is inhibited.

The present invention further pertains to a method for treating a subject having growth of an unwanted microorganism with a NSAM or an NSAM compound by administering to the subject an effective amount of the NSAM or the NSAM compound such that the subject is treated for the unwanted microorganism. The present invention even further pertains to a method for treating a subject having growth of an unwanted microorganism with a triclosan compound or with the parent triclosan compound by administering to the subject-an effective amount of the NSAM such that the subject is treated for the unwanted microorganism.

The term “subject” refers to a living animal or human in need of treatment for, or susceptible to, a condition involving an unwanted or undesirable microorganism, e.g., a particular treatment for having an unwanted pathogenic cell as defined below. In preferred embodiments, the subject is a mammal, including humans and non-human mammals such as dogs, cats, pigs, cows, sheep, goats, horses, rats, and mice. In the most preferred embodiment, the subject is a human. The term “subject” does not preclude individuals that are entirely normal with respect to having an unwanted pathogen or normal in all respects. The subject may formerly have been treated with antibiotic or antimicrobial therapy, and may be under treatment, or have been treated by antibiotic or antimicrobial therapy in the past.

The term “patient,” as used herein, refers to a human subject who has presented at a clinical setting with a particular symptom or symptoms suggesting one or more diagnoses of having an infectious disease, or having the presence of an unwanted microbial cell. A patient's diagnosis can alter during the course of disease progression, such as development of further disease symptoms, or remission of the disease, either spontaneously or during the course of a therapeutic regimen or treatment. Thus, the term “diagnosis” does not preclude different earlier or later diagnoses for any particular patient or subject. The term “prognosis” refers to assessment for a subject or patient of a probability of developing a condition associated with or otherwise indicated by presence of one or more unwanted pathogenic cells in the patient.

Methods and Uses

The environment contains a variety of microbes which are pathogenic disease organisms. These include viruses, bacteria, fungi, and protozoans, which can cause pathological damage to the subject organism if present as an unwanted cell.

The term “infectious disease” is meant to include disorders caused by one or more species of bacteria, viruses, fungi, and protozoans, species of which that are disease-producing organisms collectively referred to as “pathogens.” The term “fungi” is meant to include the yeasts. In this invention, pathogens are exemplified, but not limited to, Gram-positive bacteria such as Actinomyces bovis, Enterococcus fecalis, Hemophilus pneumoniae, Listeria monocytogenes, Mycobacterium tuberculosis, M. leprae, M smegmatis, Proprionibacterium acnes, Sarcina ventriculi, Staphylococcus aureus, S. epidermis, S. intermedias, Streptococcus hemolyticus, & pneumoniae; Gram-negative bacteria such as Campylobacter fetus, Erwinia carotovora, Flavobacterium meningosepticum, Helicobacter pylori, Hemophilus pneumoniae, H. influenzae, Klebsiella pneumonia, Neisseria gonorrhoeae, Pseudomonas aeruginosa, Shigella dysenteria, Salmonella typhi, S. paratyphi, Yersinia pestis, Escherichia coli serotype 0157, and Chlamydia species, Helicobacter species; viruses such as HIV-1, -2, and -3, HSV-I and -II, non-A non-B non-C hepatitis virus, pox viruses, rabies viruses, and Newcastle disease virus; fungi such as Candida albicans, C. tropicalis, C. krusei, C. pseudotropicalis, C. parapsilosis, C. quillermondii, C. stellatoidea, Aspergillus fumigatus, A. niger, A. nidulans, A. flavus, A. terreus, Absidia corymbifera, A. ramosa, Cryptococcus neoforms, Histoplasma capsulatum, Coccidioides immitis, Pneumocystis carinii, Rhizopus arrhizus, R. oryzae, Mucor pusillus and other fungi; and protozoa such as Entamoeba histolytica, Entamoeba coli, Giardia lamblia, G. intestinalis, Eimeria sp., Toxoplasma sp., Cryptosporidium parvum, C. muris, C. baileyi, C. meleagridis, C. wrairi, and C. nosarum. Obtaining unique epitopes from these organisms by screening proteins and by assaying peptides in vitro are commonly known to those skilled in the art.

In preferred embodiments compounds of the invention can be used to inhibit the growth of an unwanted organism, e.g., an infectious, pathogenic organism or an organism that causes spoilage or biofouling, by contacting the organism with the compound. The compound can be applied prior infection by the organism to prevent a subject from becoming infected. For example, the compounds can be used for cleaning surfaces, e.g., counter tops, instruments, or the skin of the subject, to inhibit the growth of the organism and reduce the possibility of the subject actually becoming infected with one of the organisms.

Treating or treatment of a state characterized by the presence of an unwanted cell, e.g., an unwanted pathogenic cell, e.g., an unwanted bacterium, is intended to include the alleviation of or diminishment of at least one symptom, for example, fever or inflammation, typically associated with the state. The treatment also includes alleviation or diminishment of more than one symptom. Preferably, the treatment cures, e.g., substantially eliminates, the symptoms associated with the state.

The language “therapeutically effective dose” or “therapeutically effective amount” of a compound described herein, is that amount necessary or sufficient to perform its intended function, e.g., on a surface or on or within a subject, e.g., to eradicate or inhibit growth of an unwanted pathogen, e.g., microorganism. The therapeutically effective amount can vary depending on such factors as the species or strain of the pathogen, the amount of the pathogen to be inhibited ant the manner in which the compound is to be used. One of ordinary skill in the art would be able to study the aforementioned factors and make a determination regarding the effective amount of the compound required without undue experimentation. For administration, one of ordinary skill in the art would be able to determine such amounts based on such factors as the subject's size, the severity of the subject's symptoms, and the particular composition or route of administration selected. An in vitro or in vivo assay can be here used to determine an “effective amount” of the compounds described herein to achieve inhibition of growth or proliferation of the cell by binding and inhibiting the specific target.

A “therapeutically effective dosage” is a dosage of a compound that preferably inhibits growth of an unwanted pathogenic cell, or destroys cell viability, by at least about 50%, more preferably by at least about 80%, even more preferably by at least about 90%, and still more preferably by at least about 95% relative to the absence of the compound. The ability of a compound to inhibit or kill infectious disease cells can be evaluated in an in vitro inhibitory concentration assay, or, e.g., an animal model system predictive of efficacy in infectious diseases. Alternatively, this property of a compound can be evaluated by examining the ability of the compound to inhibit in vitro by using assays well-known to the skilled practitioner. Assays include the of effect on viability of the test pathogenic cell, by assay of quantity of “colony forming units” (cfu), in the presence and absence of the compound; assay of capability to carry out a physiological process, such as cellular uptake of a metabolite; assay of uptake and incorporation of a metabolite into a macromolecule, such as a nucleic acid or protein; each assay conducted in the presence of a range of concentrations and in the absence of the compound. For compounds having a known specific target, the effective dosage to inhibit the activity of that target, such as an enzyme, can be assessed using isolated target material.

The present invention also pertains to antimicrobial soap or detergent preparations containing triclosan in amounts which are much lower than the amounts contained in the commercially available antimicrobial soap or detergent preparations. The commercially available antimicrobial soap or detergent preparations contain triclosan in higher amounts and part of the present invention includes the realization that higher amounts, e.g., than 0.3% triclosan found in Total® toothpaste, or 3 mg ml⁻¹, are not necessary for the triclosan to interact with its genomic target. The antimicrobial soap or detergent preparations contain triclosan at a concentration of less than about 500 μg per milliliter of soap or detergent preparation forming an antimicrobial soap or detergent preparation. In other embodiments, the antimicrobial soap or detergent preparations contain triclosan at a concentration of less than about, e.g., 500 μg ml⁻¹ (one ml being roughly equivalent to one gram of solid, which can be corrected by the density of the solid), less than about 100 μg ml⁻¹, less than about 50 μg ml⁻¹, e.g., less than about 10 μg ml⁻¹, less than about 5 μg ml⁻¹, less than about 1μg ml⁻¹ and e.g., less than about 0.5 μg ml⁻¹.

In addition to the above uses for the antimicrobial agents and compounds of the invention, the following uses are included: (1) a skin antiseptic: a safe, nonirritating, antimicrobial-containing preparation that prevents overt skin infection; (2) a patient preoperative skin preparation: a safe, fast-acting, broad-spectrum, antimicrobial-containing preparation that significantly reduces the number of micro-organisms on intact skin; (3) a surgical hand scrub: a safe, nonirritating, antimicrobial-containing preparation that significantly reduces the number of microorganisms on the intact skin. A surgical hand scrub should be broad-spectrum, fast-acting and persistent; (4) a health-care personnel hand wash: a safe, nonirritating preparation designed for frequent use that reduces the number of transient microorganisms on intact skin to an initial baseline level after adequate washing, rinsing and drying. If the preparation contains an antimicrobial agent, it should be broad-spectrum, fast-acting, and, if possible, persistent; (5) a skin wound cleanser: a safe, nonirritating, liquid preparation (or product to be used with water) that assists in the removal of foreign material from small, superficial wounds and does not delay wound healing; (6) a skin wound protectant: a safe, nonirritating preparation applied to small cleansed wounds that provides a protective barrier (physical, chemical, or both) and neither delays healing nor favors the growth of microorganisms; and (7) an antimicrobial soap: a soap containing an active ingredient with in vitro and in vivo activity against skin microorganisms.

The present invention also pertains to antimicrobial soap or detergent preparations containing triclosan compounds, e.g., structural analogs of triclosan, in a soap or detergent preparation. In a preferred embodiment, the structural analog of triclosan is a compound capable of inhibiting the proliferation and viability of a triclosan-resistant microbial cell.

Antimicrobial Compounds

The language “antimicrobial compound” is art-recognized and is intended to include a compound which inhibits the proliferation or viability of a microbe which is undesirable and/or which disrupts a microbial cell. The language further includes diminishment of an activity which is undesirable and associated with the microbe. Examples include antibiotics, biocides, antibacterial compounds.

The term “antibiotics” is art recognized and includes antimicrobial agents synthesized by an organism in nature and isolated from this natural source, and chemically synthesized antibiotics. The term includes but is not limited to: polyether ionophore such as monensin and nigericin; macrolide antibiotics such as erythromycin and tylosin; aminoglycoside antibiotics such as streptomycin and kanamycin; β-lactam antibiotics such as penicillin and cephalosporin; and polypeptide antibiotics such as subtilisin and neosporin. Semi-synthetic derivatives of antibiotics, and antibiotics produced by chemical methods are also encompassed by this term.

Chemically-derived antimicrobial agents such as isoniazid, trimethoprim, quinolines, and sulfa drugs are considered antibacterial drugs, although the term antibiotic has been applied to these. These agents and antibiotics have specific cellular targets for which binding and inhibition by the agent or antibiotic can be measured. For example, erythromycin, streptomycin and kanamycin inhibit specific proteins involved in bacterial ribosomal activity; penicillin and cephalosporin inhibit enzymes of cell wall synthesis; and rifampicin inhibits the β subunit of bacterial RNA polymerase. It is within the scope of the screens of the present invention to include compounds derived from natural products and compounds that are chemically synthesized.

The term “biocidal” is art recognized and includes an agent that those ordinarily skilled in the art prior to the present invention believed would kill a cell “non-specifically,” or a broad spectrum agent whose mechanism of action is unknown, e.g., prior to the present invention, one of ordinary skill in the art would not have expected the agent to be target-specific. Examples of biocidal agents include paraben, chlorbutanol, phenol, alkylating agents such as ethylene oxide and formaldehyde, halides, mercurials and other heavy metals, detergents, acids, alkalis, and chlorhexidine. The term “bactericidal” refers to an agent that can kill a bacterium; “bacteriostatic” refers to an agent that inhibits the growth of a bacterium.

In contrast to the term “biocidal,” an antibiotic or an “anti-microbial drug approved for human use” is considered to have a specific molecular target in a microbial cell. Preferably a microbial target of a therapeutic agent is sufficiently different from its physiological counterpart in a subject in need of treatment that the antibiotic or drug has minimal adverse effects on the subject.

A specific target for drug or antibiotic therapy can be ribosomal protein (S12 of the 30s ribosome); an RNA polymerase subunit (β of bacterial RNA polymerase); a cell wall (a cross-linking enzyme of a bacterial cell wall); or a DNA polymerase-associated proteins (e.g., a gyrase). In the invention here, an enzymatic component of fatty acid biosynthesis, enoyl-ACP reductase, is determined to be a specific target of an effective dose of an agent which was previously classified as a non-specific biocidal agent when used at significantly higher concentrations than the effective dose.

The term “enzyme” includes polymorphic variants that are silent mutations naturally found within the microorganism population of a strain or species. The enzymes in the preferred embodiment of the invention are fatty acid biosynthesis enzymes, preferably enoyl-ACP reductase (enoyl reductase) enzymes,.however, there is no intent to limit the invention to these enzymes. The term fatty acid biosynthesis enzymes (and its equivalent term fatty acid biosynthetases) is intended to include those components of a proteins or polypeptides capable of synthesizing fatty acids via the three-carbon intermediate, malonyl CoA. The proteins include acyl carrier protein (ACP), acetyl CoA-ACP transacetylase, malonyl CoA-transferase β-ketoacyl-ACP synthase, β-ketoacyl-ACP reductase, β-hydroxyacyl-ACP dehydratase, and enoyl-ACP reductase (Lehninger, A., et al. Principles of Biochemistry, 2nd Ed., 1993 Worth, New York, p. 642-653). The ACP of E. coli and of other organisms contains the prosthetic group 4′-phosphopantetheine, to which the growing fatty acid chain is covalently linked by a thioester bond. The term “enzymes” is art recognized for purposes of this invention and can refer to whole intact enzyme or portions or fragments thereof.

The terms “protein,” “polypeptide” and “peptide” are used interchangeably herein.

The term “variant” as used herein refers to a protein or nucleic acid molecule that is substantially similar in structure and biological activity and may substitute for the molecule of which it is a variant. Thus, provided that two molecules possess a common activity and may substitute for each other, they are considered variants as that term is used herein even if the composition or secondary, tertiary or quaternary structure of one of the molecules is not identical to that found in the other, or if the amino acid or nucleotide sequence is not identical. Variants of the ER polypeptides are intended to be included as part of this invention.

The term “fragment,” as used herein with respect to a molecule such as ER or antibody protein or a nucleic acid encoding ER, refers to a portion of a native or variant amino acid residue or nucleotide sequence. The term “fragment” includes a chemically synthesized protein fragment.

The term “antibody” as used herein is intended to include fragments thereof which are also specifically reactive with one of the components in the methods and kits of the invention. Antibodies can be fragmented using conventional techniques and the fragments screened for utility in the same manner as described above for whole antibodies. For example, F(ab)₂ fragments can be generated by treating an antibody with pepsin. The resulting F(ab)₂ fragment can be treated to reduce disulfide bridges to produce F(ab) fragments. The term “antibody” is further intended to include single chain, bispecific and chimeric molecules. The term “antibody” includes possible use both of monoclonal and polyclonal antibodies (Ab) directed against a target, according to the requirements of the application.

Polyclonal antibodies can be obtained by immunizing animals, for example rabbits or goats, with a purified form of the antigen of interest e.g., wild-type or mutant ER protein, or a fragment of the antigen containing at least one antigenic site. Conditions for obtaining optimal immunization of the animal, such as use of a particular immunization schedule, and using adjuvants e.g. Freund's adjuvant, or immunigenic substituents covalently attached to the antigen, e.g. keyhole limpet hemocyanin, to enhance the yield of antibody titers in serum, are well-known to those in the art. Monoclonal antibodies are prepared by procedures well-known to the skilled artisan, involving obtaining clones of antibody-producing lymphocyte, i.e. cell lines derived from single cell line isolates, from an animal, e.g. a mouse, immunized with an antigen or antigen fragment containing a minimal number of antigenic determinants, and fusing said clone with a myeloma cell line to produce an immortalized high-yielding cell line. Many monoclonal and polyclonal antibody preparations are commercially available, and commercial service companies that offer expertise in purifying antigens, immunizing animals, maintaining and bleeding the animals, purifying sera and IgG fractions, or for selecting and fusing monoclonal antibody producing cell lines, are available.

Specific high affinity binding proteins or peptides, that can be used in place of antibodies, can be made according to methods known to those in the art. For example, proteins that bind specific DNA sequences may be engineered (Ladner, R.C.,et. al., U.S. Pat. No.5,096,815), and proteins, polypeptides, or oligopeptides (“miniproteins”) that bind a variety of other targets, especially protein targets (Ladner, R.C., et. al., U.S. Pat. No. 5,233,409; Ladner, R. C., et.al., U.S. Pat. No. 5,403,484) may be engineered and used in the present invention for covalent linkage of a genetically replicating unit, such as a bacteriophage, displaying a library of variant peptides, to select amino acid sequences that are capable of binding to an immobilized wild-type or a mutant ER protein. The consensus of amino acid sequences of such obtained engineered binding peptides can be used as a probe of the structure of the target ER protein, and can serve as the basis of design of a peptidomimetic drug.

Antibodies and binding proteins can be incorporated into large scale diagnostic or assay protocols that require immobilizing the compositions of the present invention onto surfaces, for example in multi-well plate assays, or on beads for column purification.

Immunoassays

General techniques to be used in performing various immunoassays are known to those of ordinary skill in the art. Moreover, a general description of these procedures is provided in U.S. Pat. No. 5,051,361 which is incorporated herein by reference, and by procedures known to the skilled artisan, and described in manuals of the art (Ishikawa, E., et. al. (1988), Enzyme Immunoassay Igaku-shoin, Tokyo, NY; Harlow, E. and D. Lane, Antibodies: A Laboratory Manual, CSH Press, NY). Examples of several immunoassays are given discussed here.

Radioimmunoassays (RIA) utilizing radioactively labeled ligands, for example, antigen directly labeled with ³H, or ¹⁴C, or ¹²⁵I, measure presence of ER as antigenic material. A fixed quantity of labeled mutant ER, for example, competes with unlabeled antigen from the sample for a limited number of antibody binding sites. After the bound complex of labeled antigen-antibody is separated from the unbound (free) antigen, the radioactivity in the bound fraction, or free fraction, or both, is determined in an appropriate radiation counter. The concentration of bound labeled antigen is inversely proportional to the concentration of unlabeled antigen present in the sample. The antibody to ER can be in solution, and separation of free and bound antigen ER can be accomplished using agents such as protein A, or a second antibody specific for the animal species whose immunoglobulin contains the antibody to ER. Alternatively, antibody to ER can be attached to the surface of an insoluble material, which in this case, separation of bound and free ER is performed by appropriate washing.

Other preferred immunoassay techniques use enzyme labels such as horseradish peroxidase, alkaline phosphatase, luciferase, urease, and β-galactosidase. For example, ER conjugated to horseradish peroxidase can compete with free sample ER for a limited number of antibody combining sites present on antibodies to ER attached to a solid surface such as a microtiter plate. The anti-ER antibodies may be attached to the microtiter plate directly, or indirectly, by first coating the microtiter plate with multivalent ER conjugates (coating antigens) prepared for example by conjugating ER with serum proteins such as rabbit serum albumin (RSA). After separation of the bound labeled ER from the unbound labeled ER, the enzyme activity in the bound fraction is determined colorimetrically, for example by a multi-well microtiter plate reader, at a fixed period of time after the addition of horseradish peroxidase chromogenic substrate.

The above examples of preferred immunoassays describe the use of radioactively and enzymatically labeled tracers. Assays also may include use of fluorescent materials such as fluorescein and analogs thereof, 5-dimethylaminonaphthalene-1-sulfonyl derivatives, rhodamine and analogs thereof, coumarin analogs, and phycobiliproteins such as allophycocyanin and R-phycoerythrin; phosphorescent materials such as erythrosin and europium; luminescent materials such as luminol and luciferin; and sols such as gold and organic dyes. In one embodiment of the present invention, the biological sample is treated to remove low molecular weight contaminants.

The term “substantially pure” or “isolated” with respect to a population of genetically modified cells means that the cells contain fewer than about 20%, more preferably fewer than about 10%, most preferably fewer than about 1%, non-modified cells. The term “genetically modified” refers to mutation, including without limitation point mutation, substitution, transition, transversion, deletion, insertion, inversion and translocation mutation of nucleic acid. It includes manipulation of a recipient cell by introduction of recombinant or genetically engineered nucleic acid such as transformation and transfection.

The term “substantially pure” or “isolated” with respect to a nucleic acid or a protein means that the nucleic acid or protein is at least about 75%, preferably at least about 85%, more preferably at least about 90%, even more preferably at least about 95%, and most preferably at least about 99% free of other nucleic acids or proteins.

The term “culture medium” refers generally to any preparation suitable for cultivating living cells, preferably microorganisms. A “cell culture” refers to a cell population sustained in vitro using sterilized culture medium.

Bacterial Enoyl-ACP Reductase Mutants, Structure, and Assays

A mutation of E. coli known as envM was characterized as having a temperature-sensitive osmotic fragility phenotype (Egan, A. et al. Genet. Res. Cambr. 21: 139-152 (1973)), and was subsequently shown to be the gene for enoyl reductase and for resistance to diazoborine in this species and in Salmonella typhimurium (Tumowsky, F., et al. J. Bacteriol. 171, 6555-6565 (1989)). The envM gene had been characterized as encoding a protein involved in biosynthesis of lipopolysaccharide (Hogenauer, G. et al, Nature 293: 662-664 (1981)), and this mutation was shown to reduce virulence in E. coli clinical isolates O111:B4 and O1:K1. The envM⁺ gene was then shown to encode the FabI enoyl ACP reductase ((Turnowsky, F., et al. J. Bacteriol. 171, 6555-6565 (1989); Bergler, H., et al. J. Biol. Chem. 269, 5493-5496 (1994)).

FabI wild type and mutant proteins were expressed on plasmids in E. coli cells (Bergler, H., et al. Eur. J. Biochem. 242, 689-694 (1996)), and the proteins were overproduced, facilitating purification and assay. FabI has been engineered as an N-terminal insertion of six histidine residues, enabling purification using a Ni⁺⁺-agarose column (Qiagen, Hilden, Germany) for use in reconstitution of purified fatty acid biosynthesis components for synthesis and assay in vitro (Heath, R. et al., J. Biol. Chem. 270: 26538-26542 (1995)).

InhA of Mycobacterium smegmatis, a species susceptible to triclosan (Vischer, W. A. et al. 1974. Zbl. Bakt. Hyg., I. Abt. Orig. A 226:376-389), is 35% identical to E. coli FabI (GAP program of Genetics computer Group, Inc.[GCG]) and has enoyl reductase activity (Dessen, A. et al. 1995. Science 267:1638-1641) (Quemard, A. et al. 1995. Biochemistry 34:8235-8241). The inhA locus was originally identified by a mutation replacing serine 94 with alanine (S94A) in the gene product which caused resistance to the antitubercular drug isoniazid (Banerjee, A. et al. 1994. Science 263:227-230). Mutations conferred resistance to isoniazid, and to another anti-tuberculosis drug, ethioamide, in Mycobacterium smegmatis, M. tuberculosis, M. bovis, and M. avium (Banerjee, A., et al. Science 263, 227-230 (1994)). It is 87% identical to M. tuberculosis InhA, the three dimensional structure of which has been determined by X-ray crystallography (Dessen, A. et al. 1995. Science 267:1638-1641) in the presence of modified isoniazid (Rozwarski, D. A. et al. 1998. Science 279:98-102). X-ray crystallography of E. coli FabI (Baldock, C. et al. 1996. Science 274:2107-2110) demonstrates its structural similarity to InhA.

Structural studies involving crystallization of enoyl reductase from E. coli and X-ray crystallography of the enzyme alone and co-crystallized with diazaborine derivatives (Baldock, C., et al. Acta Cryst. D52: 1181-1184 (1996); Science 274, 2107-2110 (1996)) revealed that a covalent bond is formed between cofactor NAD and benzodiazaborine (or thienodiazaborine) through the boron atom. These analyses reveal that the drug enters the NAD cleft, and the analyses identify the residues of the cleft. Similar studies of the InhA ser94ala mutant protein reveal that isoniazid resistance is due to a decreased affinity of the mutant protein for NAD (Dessen, A., et al. Science 267, 1638-1641 (1995)). Further, covalent attachment of isoniazid to NAD in the NAD cleft can be observed (Rozwarski, D. et al. Science 279: 98-102 (1998)).

The complete fatty acid biosynthesis set of reactions can be measured in vivo using incorporation into E. coli cells of β-[3-³H] alanine into medium and long chain acyl-ACPs, which are analyzed by conformationally sensitive gel electrophoresis in 13% polyacrylamide containing 0.5 M urea (Heath, R. et al., J. Biol. Chem. 270: 26538-26542 (1995)). This in vivo assay is useful herein for screens of drug candidates among natural products and synthetic chemicals for use as antimicrobial agents, for activity that inhibits fatty acid biosynthesis, by performing the assay in the presence and absence of each compound or extract. Fatty acid synthesis can be assayed in an entirely pure in vitro system, using purified components for each reaction ((Heath, R. et al., J. Biol. Chem. 270: 26538-26542 (1995)).

Enoyl reductase activity can be measured using crude cell extracts or substantially purified enzymes by following NADH oxidation at 340 nm with a Uvikon 93310 spectrophotometer (Kontron Instruments), with 2-trans-octenoyl-ACP as a substrate (Dessen, A., et al. Science 267, 1638-1641 (1995)). This reaction can be carried out in small volumes in 96-well or 384-well multi-well plastic dishes, and can be automated for use in large-scale screens of antimicrobial agents using FabI or InhA as the specific target.

Enoyl reductase activity can also be measured in whole cells by growth with ³²Pi and measurement of incorporation into phospholipids. Following this procedure, cells are extracted with chloroform-methanol (2:1), which is then mixed with 0.25 volumes of water, and the chloroform layer is removed and analyzed for phospholipids by two-dimensional thin-layer chromatography on silica plates (Turnowsky, F., et al. J. Bacteriol. 171, 6555-6565 (1989)). Reactions can be performed in vivo in the presence and absence of drug candidates, to determine the effect on distribution of radioactivity into the spectrum of phospholipid intermediates.

Potential drug candidates can be assayed by ability to bind to an ER protein which has been immobilized on a bead or on a plastic surface, for example, the plastic of multi-well plastic dishes. A large variety of techniques for immobilization to beads and to surfaces of target proteins are described in U.S. Pat. No. 5,233,409. Candidate agents can be incubated with immobilized ER protein under appropriate conditions, for example, in the presence of NAD or NADP, and under conditions of different temperature and pH, using the known inhibitors and mutants of the invention to optimize the assay. Following incubation with the potential candidates, the immobilized ER is separated from unbound compounds, washed to remove non-specifically bound materials, and then bound materials are eluted, for example, with solutions of decreased pH, or increased detergent concentration, to obtain and analyze the specifically bound materials. Agents that are found to bind immobilized ER in this primary screen can be tested for ability to inhibit the enoyl reductase activity, and for antimicrobial activity using whole cells. Viability assays, and assays of cell lysis can also be performed in multi-well plastic dishes, in which viability is measured by cfu content following incubation in the presence and absence of drug, of dilutions of the contents of each well. Lysis can be measured by loss of optical density at, e.g., 540 nm, using an automated plate reader.

Genes, Nucleic Acids, Hybridization to Clone Homologs of ER, and Vectors

Homologs of ER proteins can be generated by mutagenesis, such as by at least one of a discrete point mutation which can give rise to a substitution, or by at least one of deletion or insertion. The present invention also is intended to encompass homologs of the ER polypeptide and mutant ER polypeptides described above. These fragments and homologs, which are biologically active in a manner which is the same or similar to the parent ER polypeptide. For example, a polypeptide or protein has ER biological activity if it can bind and reduce the double bond of an enoyl such as an octenoyl which is linked to ACP.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

As used herein, the term “gene” or “recombinant gene” refers to a nucleic acid comprising an open reading frame encoding a ER of the present invention. A “recombinant gene” refers to nucleic acid encoding a ER protein encoded by a gene that has been engineered by recombinant techniques. The nucleotide sequence encoding FabI is shown in SEQ ID NO: 2.

The term “vector” refers to a nucleic acid molecule capable of transporting another nucleic acid to which it has been linked. The term “expression vector” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions.

The terms “transformation” and “transfection” mean the introduction of a nucleic acid, e.g., an expression vector, into a recipient or “host” cell. The term “transduction” means transfer of a nucleic acid sequence, preferably DNA, from a donor to a recipient cell, by means of infection with a virus previously grown in the donor, preferably a bacteriophage, preferably phage P1.

The term “gene product” includes an RNA molecule transcribed from a gene, or a protein translated from the RNA transcribed from the gene.

Vectors capable of directing the expression of genes to which they are operatively linked are referred to herein as “expression vectors”. Expression vectors for expression of the er gene and capable of replication in a cell of a bacterium, such as an Escherichia, a Bacillus, a Streptomyces, a Streptococcus, or in a cell of a simple eukaryotic fungus such as a Saccharomyces or, a Pichia, or in a cell of a eukaryotic organism such as an insect, a bird, a mammal, or a plant, are within the present invention. Such vectors may carry functional replication-specifying sequences (replicons) both for a host for expression, for example a Streptomyces, and for a host, for example, E. coli, for genetic manipulations and vector construction. See e.g. U.S. Pat. No. 4,745,056. Suitable vectors for a variety of organisms are described in Ausubel, F. et al., Short Protocols in Molecular Biology, Wiley, New York (1995), and for example, for Pichia, can be obtained from Invitrogen (Carlsbad, Calif.).

“Transcriptional regulatory sequence” is a generic term to refer to DNA sequences, such as initiation signals, enhancers, and promoters, which induce or control transcription of protein coding sequences with which they are operably linked. In preferred embodiments, transcription of a recombinant ER gene, a marRAB sequence or acrAB sequence, is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the ER protein. Exemplary regulatory sequences are described in Goeddel; Gene Expression Technology: Methods in Enzymology 185, Academic Press, San Diego, Calif. (1990). For instance, any of a wide variety of expression control sequences, that control the expression of a DNA sequence when operatively linked to it, may be used in these vectors to express DNA sequences encoding the ER proteins of this invention.

“Homology” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous or identical at that position. A degree of homology between sequences is a function of the number of matching or identical positions shared by the sequences.

“Cells,” “host cells,” “recipient cells,” or “sensitive recipient cells,” are terms used interchangeably herein. It is understood that such terms refer not only to the particular subject cell but to the progeny or potential progeny of such a cell. Recipient cells are “sensitive” for the drug that is used to select for the particular drug-resistant trait of interest encoded by the transducing or transforming nucleic acid, e.g., in the invention, the cell can be sensitive to one or more of ampicillin, kanamycin, or triclosan.

In one embodiment, the invention includes a nucleic acid which encodes a peptide having enoyl reductase enzyme activity, e.g., Fab1 or InhA. Preferably, the nucleic acid is a PCR product molecule comprising at least a portion of the nucleotide sequence represented in SEQ ID NO: 1 or SEQ ID NO: 2 from nucleotide (nt) 404 to 1189, or a homolog or variant thereof.

Preferred nucleic acids encode a bacterial FabI protein comprising an amino acid sequence at least 50% homologous, more preferably 75% homologous and most preferably 80%, 90%, or 95% homologous with an amino acid sequence shown in one of SEQ ID NO: 3. Nucleic acids which encode polypeptides having an activity of a FabI protein and having at least about 90%, more preferably at least about 95%, and most preferably at least about 98-99% homology with a sequence shown in SEQ ID NO: 2 are within the scope of the invention.

Preferred nucleic acids encode a bacterial InhA protein comprising an amino acid sequence at least 50% homologous, more preferably 75% homologous and most preferably 80%, 90%, or 95% homologous with an amino acid sequence shown in one of SEQ ID NO: 12. Nucleic acids which encode polypeptides having an activity of a InhA protein and having at least about 90%, more preferably at least about 95%, and most preferably at least about 98-99% homology with a sequence shown in SEQ ID NO: 11 are within the scope of the invention.

Another aspect of the invention provides a nucleic acid which hybridizes under high stringency conditions to a “probe”, which is a nucleic acid molecule which binds specifically to a nucleic acid molecule encoding an ER enzyme. A suitable probe is at least 12 nucleotides in length, is single-stranded, and is labeled, for example, radiolabeled or fluorescently labeled. Appropriate moderate stringency conditions which promote DNA hybridization, for example, 6.0 × sodium chloride/sodium citrate (SSC) at about 45° C., are followed by successive washes of increased stringency, e.g., 2.0 × SSC at 50° C., and are known to those skilled in the art or can be found in Current Protocols in Molecular Biology, John Wiley & Sons, N.Y. (1989), 6.3.1-6.3.6. Other suitable stringency conditions include selecting the salt concentration in the wash step from a low stringency of about 2.0 × SSC at 50° C., and then using a wash of a high stringency condition, of about 0.2 × SSC at 50° C. In addition, the temperature in the wash step can be increased from low stringency conditions at room temperature, about 22° C., to high stringency conditions at about 65° C. Exemplary probes for DNA sequencing and for PCR analysis of FabI are shown in SEQ ID NOS: 4-10.

Conditions for hybridizations are largely dependent on the melting temperature that is observed for half of the molecules of a substantially pure population of a double-stranded nucleic acid, a parameter known as the Tm which is the temperature in ° C. at which half the molecules of a given sequence are melted or single-stranded. For nucleic acids of sequence 11 to 23 bases, the Tm can be estimated in degrees C. as 2(number of A+T residues)+4(number of C+G residues). Hybridization or annealing of the probe to the nucleic acid being probed should be conducted at a temperature lower than the Tm, e.g., 15° C., 20° C., 25° C. or 30° C. lower than the Tm. The effect of salt concentration (in M of NaCl) can also be calculated, see for example, Brown, A., “Hybridization” pp. 503-506, in The Encyclopedia of Molec. Biol., J. Kendrew, Ed., Blackwell, Oxford (1994).

Fragments of the nucleic acids encoding ER proteins are within the scope of the invention. As used herein, a fragment of the nucleic acid encoding a portion of a ER protein refers to a nucleic acid molecule having fewer nucleotides than the nucleotide sequence encoding the entire amino acid sequence of ER protein but which nevertheless encodes a peptide having the biological activity, e.g., enoyl-ACP reductase activity. Nucleic acid fragments within the scope of the present invention include those capable of hybridizing under high stringency conditions with nucleic acids from other species for use in screening protocols to detect ER homologs and naturally occurring polymorphic alleles.

Useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. A useful translational enhancer sequence is described in U.S. Pat. No. 4,820,639.

It should be understood that the design of the expression vector may depend on such factors as the choice of the host cell to be transformed and/or the type of protein desired to be expressed. In one embodiment, the expression vector includes a recombinant gene encoding a peptide having an activity of a ER protein. Such expression vectors can be used to transfect cells and thereby produce proteins or peptides, including fusion proteins or peptides, encoded by nucleic acids as described herein.

As used herein, a “derivative” or “analog” of an antimicrobial compound (e.g., a peptide) refers to a form of that compound in which one or more reaction groups on the compound have been derivatized with a substituent group (e.g., alkylated or acylated peptides). As used herein an “analog” of a compound refers to a compound that retains chemical structures necessary for functional activity yet that also contains certain chemical structures that differ. An example of an analog of a naturally-occurring peptide is a peptide that includes one or more non-naturally-occurring amino acids. As used herein, a “mimetic” of a compound refers to a compound in which chemical structures necessary for functional activity have been replaced with other chemical structures that mimic the conformation. Examples of peptidomimetics include peptidic compounds in which the peptide backbone is substituted with one or more benzodiazapine molecules (see e.g., James, G. L. et al., (1993) Science 260:1937-1942) and “retro-inverso” peptides (see U.S. Pat. No. 4,522,752 by Sisto), described further below. A “residue” refers to an amino acid in a position in a peptide, or an amino acid mimetic incorporated in the peptide compound by an amide bond or amide bond mimetic. Approaches to designing peptide derivatives, analogs and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

An “amino acid mimetic” refers to a moiety, other than a naturally occurring amino acid, that conformationally and functionally serves as a substitute for a particular amino acid in a peptide-like compound without adversely interfering to a significant extent with the function of the compound (e.g., inhibition of ER). In some circumstances, substitution with an amino acid mimetic may actually enhance properties of the inhibitor (e.g., interaction of the inhibitor with ER). Examples of amino acid mimetics include D-amino acids. Peptides substituted with one or more D-amino acids may be made using well known peptide synthesis procedures. The effect of amino acid substitutions with D-amino acids and other peptidomimetics can be tested using assays as described herein.

The peptide analogs or mimetics of the invention include isosteres. The term “isostere” as used herein refers to a sequence of two or more residues that can be substituted for a second sequence because the steric conformation of the first sequence fits a binding site specific for the second sequence. The term specifically includes peptide backbone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including Ψ[CH₂S], Ψ[CH₂NH], Ψ[C(S)NH₂], Ψ[NHCO], Ψ[C(O)CH₂], and Ψ[CH═CH]. In the nomenclature used above, Ψ indicates the absence of an amide bond. The structure that replaces the amide group is specified within the brackets. Other examples of isosteres include peptides substituted with one or more benzodiazapine molecules (see e.g., James, G. L. et al. (1993) Science 260:1937-1942)

Other possible modifications include an N-alkyl (or aryl) substitution (Ψ[CONR]), backbone crosslinking to construct lactams and other cyclic structures, or retro-inverso amino acid incorporation (Ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if a parent peptide is Thr-Ala-Tyr, the retro modified form is Tyr-Ala-Thr, the inverso form is thr-ala-tyr, and the retro-inverso form is tyr-ala-thr using lower case letters to refer to D-amino acids. Compared to the parent peptide, a retro-inverso peptide has a reversed backbone while retaining substantially the original spatial conformation of the side chains, resulting in a retro-inverso isomer with a topology that closely resembles the parent peptide and is able to bind the selected cysteine protease. See Goodman et al. “Perspectives in Peptide Chemistry” pp. 283-294 (1981). See also U.S. Pat. No. 4,522,752 by Sisto for further description of “retro-inverso” peptides.

Pharmaceutical Compositions

The invention provides pharmaceutically acceptable compositions which include a therapeutically-effective amount or dose of an antimicrobial compound, e.g., triclosan, and one or more pharmaceutically acceptable carriers (additives) and/or diluents. A composition can also include a second antimicrobial agent, e.g., an inhibitor of an efflux pump.

As described in detail below, the pharmaceutical compositions can be formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream, foam, or suppository; or (5) aerosol, for example, as an aqueous aerosol, liposomal preparation or solid particles containing the compound.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions of the present invention may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a sucrose octasulfate and/or an antibacterial or a contraceptive agent, drug or other material other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” according to this invention is defined as an area of tissue that covers external surfaces of a body, or which and lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Such compositions can be particularly useful, for example, for treatment or prevention of an unwanted celi, e.g., vaginal Neisseria gonorrhea, or infections of the oral cavity, including cold sores, infections of eye, the skin, or the lower intestinal tract. Standard composition strategies for topical agents can be applied to the antimicrobial compounds, e.g., triclosan or a pharmaceutically acceptable salt thereof in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate, and which is solid at room temperature, but liquid at body temperature and, therefore, will melt in the rectum or vaginal cavity and release the active agent.

Compositions which are suitable for vaginal administration also include pessaries, tampons, creams, gels, pastes, foams, films, or spray compositions containing such carriers as are known in the art to be appropriate. The carrier employed in the sucrose octasulfate /contraceptive agent should be compatible with vaginal administration and/or coating of contraceptive devices. Combinations can be in solid, semi-solid and liquid dosage forms, such as diaphragm, jelly, douches, foams, films, ointments, creams, balms, gels, salves, pastes, slurries, vaginal suppositories, sexual lubricants, and coatings for devices, such as condoms, contraceptive sponges, cervical caps and diaphragms.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolatum. Exemplary ophthalmic compositions include eye ointments, powders, solutions and the like.

Powders and sprays can contain, in addition to sucrose octasulfate and/or antibiotic or contraceptive agent(s), carriers such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Compositions of the invention can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antimicrobial agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspension. These suspensions may be formulated according to techniques known in the art using suitable dispersing or wetting agents and suspending agents. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The antimicrobial agent or a pharmaceutically acceptable salt thereof will represent some percentage of the total dose in other dosage forms in a material forming a combination product, including liquid solutions or suspensions, suppositories, douches, enemas, gels, creams, emulsions, lotions slurries, soaps, shampoos, detergents, powders, sprays, lipsticks, foams, pastes, toothpastes, ointments, salves, balms, douches, drops, troches, lozenges, mouthwashes, rinses and others. Creams and gels for example, are typically limited by the physical chemical properties of the delivery medium to concentrations less than 20% (e.g., 200 mg/gm). For special uses, far less concentrated preparations can be prepared, (e.g., lower percent formulations for pediatric applications). For example, the pharmaceutical composition of the invention can comprise sucrose octasulfate in an amount of 0.001-99%, typically 0.01-75%, more typically 0.1-20%, especially 1-10% by weight of the total preparation. In particular, a preferred concentration thereof in the preparation is 0.5-50%, especially 0.5-25%, such as 1-10%. It can be suitably applied 1-10 times a day, depending on the type and severity of the condition to be treated or prevented.

Given the low toxicity of an antimicrobial agent or a pharmaceutically acceptable salt thereof over many decades of use as a biocide [W. R. Garnett, Clin. Pharm. 1:307-314 (1982); R. N. Brogden et al., Drugs 27:194-209 (1984); D. M. McCarthy, New Eng J Med., 325:1017-1025 (1991), an upper limit for the therapeutically effective dose is not a critical issue. For most forms of triclosan the minimum amount present in the materials forming combinations of this invention that is effective in treating or preventing bacterial disease due to direct interaction with the organism should produce be less than 0.1 μg ml⁻¹, less than 0.5 μg ml⁻¹, preferably less than 1μg ml⁻¹, even more preferably less than less than 5 μg ml⁻¹, and most preferably less than 10μg ml⁻¹.

For prophylactic applications, the pharmaceutical composition of the invention can be applied prior to physical contact. The timing of application prior to physical contact can be optimized to maximize the prophylactic effectiveness of the compound. The timing of application will vary depending on the mode of administration, the epithelial surface to which it is applied, the surface area, doses, the stability and effectiveness of composition under the pH of the epithelial surface, the frequency of application, e.g., single application or multiple applications. Preferably, the timing of application can be determined such that a single application of composition is sufficient. One skilled in the art will be able to determine the most appropriate time interval required to maximize prophylactive effectiveness of the compound.

One of ordinary skill in the art can determine and prescribe the effective amount of the pharmaceutical composition required. For example, one could start doses at levels lower than that required in order to achieve the desired therapeutic effect and gradually increase the dosage until the desired effect is achieved. In general, a suitable daily dose of a composition of the invention will be that amount of the composition which is the lowest dose effective to produce a therapeutic effect. Such an effective dose will generally depend upon the factors described above. It is preferred that administration be intravenous, intracoronary, intramuscular, intraperitoneal, or subcutaneous.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, NY (1995)); DNA Cloning, Volumes I and II (D. N. Glover ed., 1985); Oligonucleotide Synthesis(M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The invention is further illustrated by the following examples, which should not be construed as further limiting. The contents of all references, pending patent applications and published patents, cited throughout this application are hereby expressly incorporated by reference.

### EXAMPLES

The following methodology described in the Materials and Methods section was used throughout Examples 1-9.

Materials and Methods

Isolation of Triclosan Resistant Mutants

All experiments were performed at 37° C. using LB broth or agar (Ausubel et al. supra) (Short Protocols in Molecular Biology, 3rd Ed., ed. by Ausubel, F. et al. (Wiley, NY (1995)). Independent cultures of an E. coli K 12 strain, AG100 (George, A. M. & Levy, S. B. J. Bacteriol. 155, 531-540 (1983)) were grown overnight to stationary phase at 37° C. and 10⁸colony-forming-units (cfu) from each culture were plated onto agar containing 0.2 or 0.3 μg ml⁻¹ triclosan having the structure 2,4,4′-trichlor-2′-hydroxydiphenyl ether, CAS # 3380-34-5 (trade name Irgasan® DP300, Ciba CH3565, available from Ciba Specialty Chemicals Corp., Greensboro, N.C.; stock solutions dissolved in ethanol).

After incubation for 24-48 h, one resistant colony from each of six cultures was purified on agar containing triclosan. Resistance to triclosan was quantitated using serial dilution plates with 2.0 fold steps of increasing concentrations of triclosan. Five μl of log phase cells containing approximately 4×10⁴ cfu was applied as a spot to the dilution plates. The lowest triclosan concentration which inhibited growth after 20 h defined the minimal inhibitory concentration (MIC). Inhibition of growth rate was determined in broth culture by adding triclosan at various concentrations to log phase cells which had reached an absorbance (A₅₃₀) of 0.1 and determining the effect on the rate of change of absorbance 1 h later; lysis was identified by a loss of absorbance (about 50%) accompanied by a 4-5 log loss in viable cfu per A₅₃₀ unit.

A chromosomal library was prepared from mutant AGT11 by cloning 1-7 kb Sau3aI partial digestion fragments into the BamHI site of the tet gene in pBR322, transforming into strain DH5α (Gibco/BRL, Bethesda Md.), and selecting on ampicillin (Sigma, St. Louis, Mo.). Approximately 16,000 transformants were pooled to form the library, and the clones encoding triclosan resistance were found by plating about 80,000 cfu from the library on 0.3 μg ml⁻¹ triclosan.

Other Methods and Strains.

Chromosomal DNA was prepared using a Puregene kit (Gentra Systems, Minneapolis, Minn.). PCR products of AGT23 and AGT25 were generated for sequencing using Taq DNA polymerase (Gibco) and oligonucleotide pairs LM011, SEQ ID NO: 4, and LM010, SEQ ID No. 5 (respectively, nt 160-179 and 1168-1149). The numbering system for fabI of Bergler, H., et al. (J. Gen. Microbiol. 138, 2093-2100 (1992)), in which the fabI gene is nt 404-1189; see SEQ ID No. 1) was used. Other sequencing primer pairs were LM019, SEQ ID NO: 6, and LM020, SEQ ID NO: 7 (respectively, nt 1291-1275 and 745-762). The same oligonucleotides were used for sequencing the products. Junctional DNA in pLYT6 and pLYT8 was sequenced using oligonucleotide BR346, SEQ ID NO: 9 (nt 346-357 in pBR322, in which the BamHI site is at nt 375 (see the catalog of New England Biolabs, Beverly, Mass.). The fabI gene in pLYT8 was sequenced using LM010, LM 019, and LM011. pLYT27 was sequenced using LM015 (nt 875-856; see SEQ ID NO: 8) and LM021, SEQ ID NO: 10 (in pBR322, nt 4068-4086).

Strain JZM120 (ΔacrAB::kan; Ma, D., et al. Molec. Microbiol. 16, 45-55 (1995)) (from H. Nikaido) served as the donor strain for bacteriophage P1-mediated transduction (Provence, D. L. & Curtiss, R. I. in Methods for General and Molecular Bacteriology eds. Gerhardt, P., Murray, R. G. E., Wood, W. A. & Kreig, N. R. 317-347 American Society for Microbiology, Washington, D. C., (1994); Miller, J. Experiments in Molecular Genetics(Cold Spring Harbor Press, Cold Spring Harbor, N.Y. (1972)) in the inactivation of the acrAB locus in other strains. Strain AG100/kan (ΔmarCORAB; Maneewannakul, K., et al. Antimicrobial Agents Chemother. 40, 1695-1698 (1996)) similarly was used to inactivate the mar locus. Because of the reported low aqueous solubility of triclosan (10 mg ml⁻¹; Ciba-Geigy, Irgasan® DP300 material safety data sheet No. 235 (1996)), some MIC experiments were performed in hypersusceptible host AG100A (which is AG100 ΔacrAB::kan) to reduce the triclosan concentration required. Strain AGT11K is AGT11ΔacrAB::kan.

### EXAMPLE 1

### Isolation of Mutants Resistant to Triclosan

A genetic approach was used to find the mechanism of triclosan action in Escherichia coli. Mutants resistant to triclosan were isolated and then the resistance locus was cloned and identified. The roles of the AcrAB multidrug efflux pump and of its positive regulator MarA in the susceptibility of strains to triclosan were then investigated.

Six independent triclosan resistant mutants of E. coli K12 strain AG100 were isolated as described in Methods. The MICs ranged from 1.7 to 145 times the 0.28 μg ml⁻¹ MIC of the parental strain (Table 1, MIC column 1). Further, triclosan-resistant E. coli strain AGT11 had several times the isoniazid resistance of the isogenic parent AG100(determined in the presence of 250 μM hydrogen peroxide to reduce the inherently high resistance of E. coli to isoniazid).

### EXAMPLE 2

### The Role of Two Loci, acrAB and marRAB, in Triclosan Resistance

The acrAB operon in Escherichia coli encodes a multidrug efflux pump which provides intrinsic resistance to many diverse compounds including antibiotics and disinfectants (Nikaido, H. J. Bacteriol. 178, 5853-5859 (1996)). This operon can be up-regulated by MarA (Ma, D., et al. Molec. Microbiol. 16, 45-55 (1995), a transcriptional activator encoded by the marRAB operon involved in multiple antibiotic resistance (Alekshun, M. N., et al. Antimicrob. Agents Chemother. 41, 2067-2075 (1997)).

Mar mutants overexpressing the mar operon were twice as resistant to triclosan as the parental strain AG100. In the mutants selected on triclosan, inactivation of marRAB had little effect upon triclosan resistance (Table 1, MIC column 2) in comparison to these mutants having marRAB⁺ activity (Table 1, MIC column 1).

TABLE 1

| | | MIC of strain divided | | |
|---|---|---|---|---|
| | Mutation in | by MIC of AG1000* | | |
| Strain | fabI | none** | mar** | acrAB** |
| AG100 | none | 1 | 0.71 | 0.063 |
| AGT7 | NI | 1.7 | 1.7 | 0.071 |
| AGT8 | NI | 4 | 3.4 | 0.25 |
| AGT9 | NI | 2.3 | 2.3 | 0.32 |
| AGT21 | G93V | 145 | 145 | 11.4 |
| AGT23 | M159T | 11.4 | ND | 1.7 |
| AGT25 | F203L | 4.6 | ND | 0.57 |

Relative triclosan resistance of mutants selected upon triclosan and effect of inactivation of the marRAB and acrAB loci. Minimal inhibitory concentrations (MICs) were determined in duplicate on the complete set of strains by the agar dilution technique as described in Methods, and the mean values are presented as ratios to the MIC of wild type strain AG100. The greatest average deviation from the mean, seen for one strain, was 33%. NI, not identified, mutation in fabI based on P1 transduction experiments

*The MIC of AG100 was 0.28±0.04 μg ml⁻¹

**Inactivated locus

Inactivation of acrAB increased the susceptibility of all strains (including that of the triclosan susceptible parent AG100) approximately 7-24 fold (Table 1, MIC column 3). Increased triclosan resistance of fabI acrAB mutants was observed compared to acrAB inactivated in the fabI⁺ AG100. The AcrAB multidrug pump was an effective exporter of triclosan but was not the basis of the enhanced resistance in the fabI mutants.

Loss of the AcrAB multidrug efflux pump presumably permits a greater concentration of triclosan within the cytoplasm of the cell, where FabI is located (Cronan, J. E., Jr. & Rock, C. O. in Escherichia coli and Salmonella: Cellular and Molecular Biology (ed. Neidhardt, F. C.) 612-636 (ASM Press, Washington, D.C., 1996)), resulting in the observed increase in susceptibility of cells to the drug.

### EXAMPLE 3

### Transduction and Cloning of Triclosan Resistance

The triclosan resistance phenotype of mutant AGT11 could be transduced to recipient strain AG100A using P1 phage (Provence, D. L., et al. Methods for General and Molecular Bacteriology, eds. Gerhardt, P., et al. pp. 317-347, American Society for Microbiology, Washington, D. C., 1994); Miller, J. Experiments in Molecular Genetics (Cold Spring Harbor Press, Cold Spring Harbor, N.Y. (1972)), indicating that the mutation conferring the resistance phenotype might lie in one clonable locus.

A genomic Sau3AI library from strain AGT11 was prepared in plasmid pBR322, and transformed into strain DH5α (see Methods). Clones mediating triclosan resistance were obtained in the library at a frequency of about 1 in 2500 transformants. Ten clones, named pLYT1 through pLYT10, were isolated and screened. The plasmids isolated from these clones bore inserts of various sizes. Digestion of plasmids with HindIII and SphI revealed that all plasmid clones had a fragment of approximately 1530 bp. All clones gave the same MIC (about 4 μg ml⁻¹, measured in hypersensitive strain AG100A), compared to 0.005-0.02 μg ml⁻¹ for the vector alone in the same host. The level of resistance by the mutation present as a single copy on the chromosome (for example, the MIC of strain AGT11K) was 2 to 4 μg ml⁻¹.

### EXAMPLE 4

### Identification of the Triclosan Resistance Gene by Sequence

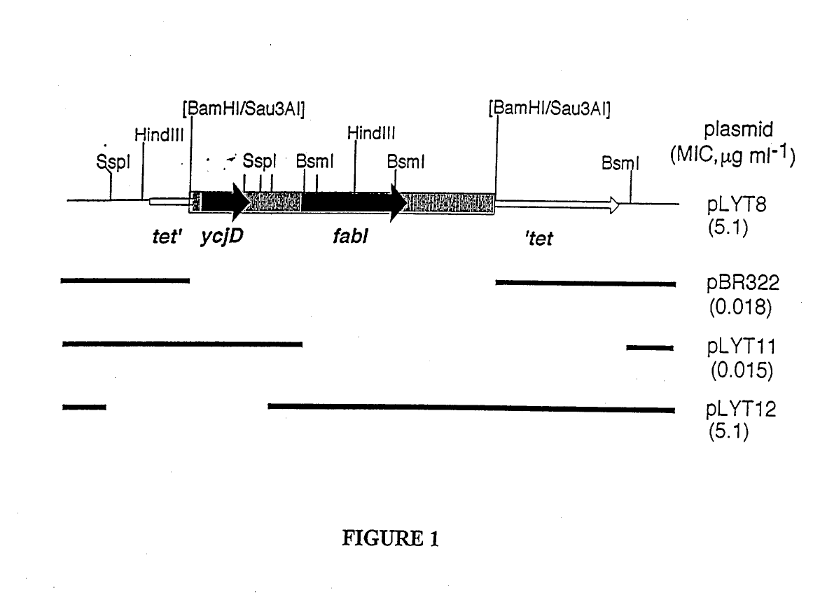

The junctional DNA sequences present in two clones with inserts of different sizes, pLYT6 and pLYT8, were obtained using a pBR322 primer. The sequences were compared to those deposited in the E. coli genomic database. The sequence data and the sizes of the inserts showed that each insert bore the fabI gene together with an upstream putative open reading frame ycjD (FIG. 1, pLYT8). The 1530 bp fragment possessed by all tested clones proved to be a HindIII fragment extending from the HindIII site in the vector to a HindIII site in the middle of fabI. The inserts in these clones may all have had the same orientation (that of the tet gene in the vector).

To see which gene, ycjD or fabI, was able to confer triclosan resistance, a BsmI fragment containing half of fabI was deleted from pLYT8, producing plasmid pLYT11 (FIG. 1). This deletion produced loss of triclosan resistance (FIG. 1). Further, an SspI fragment which included the tet promoter and all of ycjD was deleted from pLYT8, producing plasmid pLYT12 (FIG. 1). This deletion had no effect on triclosan resistance (FIG. 1). These results show that triclosan resistance was conferred by fabI gene. Further, transcription from the tet promoter was not required for expression of triclosan resistance.

### EXAMPLE 5

### Substitution Mutation in fabI Cause Triclosan Resistance

The fabI gene encodes enoyl ACP reductase, an enzyme involved in the synthesis of fatty acids (Cronan, J. E., Jr., et al. in Escherichia coli and Salmonella: Cellular and Molecular Biology, ed. Neidhardt, F. C., 612-636 ASM Press, Washington, D.C., (1 996)) which reduces a double bond using NADH or NADPH (Bergler, H., et al. Eur. J Biochem. 242, 689-694 (1996)). To determine if mutations were present in fabI, the entire fabI gene of pLYT8 from residues 190 to 1260 was sequenced (residues are identified using the numbering system of Bergler (Bergler, H., et al. J. Gen. Microbiol. 138, 2093-2100 (1992)), including the upstream “BoxC” region (Bergler, H., et al. J. Gen. Microbiol. 138, 2093-2100 (1992)).

The sequence obtained was compared to that of fabI in the database (shown in SEQ ID NO: 1, from nt 404-1189). Codon 93 in fabI was found to have mutated from ggt to gtt, thereby substituting the wild type glycine at residue 93 of the fabI protein (SEQ ID NO:2) with valine in the mutant enzyme.

### EXAMPLE 6

### Demonstration of gly 93val Mutation Role in Triclosan Resistance by Backcross

To determine whether the mutation is the cause of resistance, or whether it is a mere sequence variant unique to strain AG100 and that pLYT8 resistance was due to the presence of a wild-type fabI gene in multicopies on the plasmid, a “backcross” of wild-type DNA into the mutant was performed. This cross tests whether the real chromosomal mutation leading to triclosan resistance in mutant strain AGT11 had been identified. The mutation gly93val affects the same residue as the gly93ser mutation in the FabI protein which was shown to cause resistance to the heterocyclic inhibitor diazaborine (Bergler, H., et al. J. Gen. Microbiol. 138, 2093-2100 (1992); Tumowsky, F., et al. J. Bacteriol. 171, 6555-6565 (1989)).

The mutation-bearing 606 bp SspI-HindIII fragment of pLYT12 was replaced with the wild type SspI-HindIII counterpart from a PCR product of chromosomal DNA from parental strain AG100. The 606 bp region of the resulting plasmid, pLYT27, was sequenced to confirm that the DNA derived from AG100 in fact carried the wild type sequence identical to that in the database. The triclosan MIC measured for pLYT27 in host AG100A was 20-30 fold greater than that for vector pBR322 itself, showing a clear multicopy effect. However, this increase in resistance was notably less than the 280-340 fold increased MIC measured for pLYT8 and pLYT12, the plasmids bearing the gly93val mutation (FIG. 1). Therefore the gly93val mutation was responsible for triclosan resistance in the original mutant AGT11, as its replacement with the wild-type allele confered triclosan sensitivity.

How triclosan might inhibit FabI is informed by studies on diazaborine, a boron-containing, heterocyclic inhibitor of E. coli and Salmonella typhimurium FabI. Diazaborine resistance results from a gly93ser mutation (Bergler, H., et al. J. Gen. Microbiol 138, 2093-2100 (1992); Tumowsky, F., et al. J. Bacteriol. 171, 6555-6565 (1989)), similar to the gly93val mutation shown here to cause a high level of triclosan resistance. In the wild type FabI enzyme, binding of diazaborine is dependent upon the presence of the cofactor NAD(H) (Kater, M. M., et al. Plant Molec. Biol. 25, 771-790 (1994); Bergler, H., et al. J. Biol. Chem. 269, 5493-5496 (1994)). The gly93ser mutation reduces the binding of diazaborine to the enzyme (Bergler, H., et al. J. Biol. Chem. 269, 5493-5496 (1994)) and also results in lowered specific activity of the enzyme (Bergier, H., et al. Eur. J. Biochem. 242, 689-694 (1996)).

Of the triclosan-resistant fabI mutants isolated here, the growth rate in broth of mutant AGT11 was about 40% less, and that of mutant AGT23 about 15% less, than that of the wild type parent. These data show that the FabI enzyme in the mutants is a less active enzyme than that of thefabI⁺ parent.

### EXAMPLE 7

### Sequences of fabI in Other Triclosan Resistant Mutants

PCR products of the entire fabI gene of two other triclosan resistant mutants, AGT23 and AGT25, were synthesized using chromosomal DNA as template. The sequence of the PCR product of strain AGT23 revealed a single point mutation (atg became acg), leading to replacement of methionine 159 by threonine. Strain AGT25 had a single point mutation (ttc became ctc), leading to replacement of phenylalanine 203 by leucine. Thus, mutations in fabI were responsible also for the triclosan resistance phenotype of strains AGT11, AGT23, and AGT25.

Mutations at residues 93, 159, and 203 led to triclosan resistance, correlating with the recently-determined crystal structure of wild type E. coli FabI protein (Baldock, C., et al. Science 274, 2107-2110 (1996)). This structure shows that these three residues line the cleft of FabI in which NAD⁺ (and diazaborine) bind. The structure also shows NAD⁺ and diazaborine covalently linked to each other via the boron of the latter.

Triclosan, diazaborine and isoniazid can interact in a related manner with enoyl-ACP reductases as indicated by the following facts. InhA, the gene encoding enoyl-ACP reductase of Mycobacterium tuberculosis, has 40% sequence identity with E. coli FabI (Banerjee, A., et al. Science 263, 227-230 (1994)). A mutation of serine 94 to alanine is associated with isoniazid resistance in both M. smegmatis and M. tuberculosis (Banerjee, A., et al. Science 263, 227-230 (1994)). In this organism the crystal structures of both the wild type and mutant InhA proteins were determined, showing that they have different conformations in the NAD binding site near amino acid residue 94, leading in the mutant to decreased affinity for NAD, and thus for the inhibitor (Dessen, A., et al. Science 267, 1638-1641 (1995)).

Further, triclosan-resistant E. coli strain AGT11 had several times the isoniazid resistance of the isogenic parent AG100(determined in the presence of 250 μM hydrogen peroxide to reduce the inherently high resistance of E. coli to isoniazid). Although M. smegmatis is susceptible to triclosan, M. tuberculosis is not sensitive (Vischer, W. A., et al. Zbl. Bakt. Hyg., I. Abt. Orig. A 226, 376-389 (1974)).

The related crystal structure for another homologous enoyl reductase, that of the rape seed oil plant, Brassica napus, has also been determined (Rafferty, J. B., et al. Structure 3, 927-938 (1995)). Diazaborine and triclosan both have two unsaturated rings but otherwise are structrually different, and isoniazid has a single ring. Two of these structures can covalently bind with NADH when present together in the ER site for reducing agents.

### EXAMPLE 8

### Chromosomal Mapping of Triclosan Resistance to Min 28.5

Linkage of the triclosan resistance locus in three unsequenced mutants AGT7, AGT8, and AGT9 (and in the sequenced mutants AGT11 and AGT23 provided as controls) was used to map this gene to min 28.5, the location of fabI. This was done using P1 transduction of zci-3118::Tn10kan at approximately min 28.5 (Singer, M., et al. Microbiol. Rev. 53, 1-24 (1989)), from a wild type donor strain into each of the mutants. Of 10 kanamycin resistant transductants analysed for each mutant, 3 to 6 had acquired triclosan sensitivity. These data support triclosan resistance being due to mutations in the fabI gene.

These findings together suggested that triclosan most likely acts upon wild type FabI, thereby inhibiting synthesis of fatty acids and consequently of lipids, lipopolysaccharides, and membranes, leading to decreased growth.

### EXAMPLE 9

### Lysis of Cells by Triclosan Occurs at Higher Concentrations than Inhibition of the FabI Primary Target

Triclosan lyses cells of E. coli (Regos, J., et al. Zbl. Bakt. Hyg., I. Abt. Orig. A 226, 390-401 (1974)) and Porphyromonas gingivalis (Cummins, D. J Clin. Periodont. 18,455-461 (1991)).

Triclosan here was found to cause a loss of absorbance in broth cultures of growing susceptible E. coli, accompanied by a decrease in recoverable viable cells due to cell lysis, at concentrations of triclosan higher than those which affected the growth rate. To inhibit the growth rate 50%, about 0.15 μg ml⁻¹ triclosan was required for wild type strain AG100, about 0.02 μg ml⁻¹ triclosan for AG100A (deleted of acrAB), and about 1 μg ml⁻¹ for triclocan-resistant mutant derivative AGT11K (gly93val, otherwise isogenic to AG100A). On the other hand, the amount of triclosan required to give lysis was 2-8 μg ml⁻¹ for these strains. However, strain AGT11 fabI gly93val, otherwise isogenic to AG 100, did not display lysis even when triclosan up to a level of 256 μg ml⁻¹ was added. This indicates that FabI is involved in protection from cell lysis even at high concentrations of triclosan. Therefore mutations to triclosan resistance in fabI affect both the FabI inhibitory activity of this agent and also its lysis activity in bacteria. These data indicate that cells that are currently resistant to triclosan can be made susceptible to lysis by use of one or more additional agents specific for an efflux pump.

Further, as the data in Table 1 show that acrAB deletions can restore triclosan sensitivity and lysis to the fabI mutants, cells that are currently resistant to triclosan can be made susceptible to lysis by use of one or more agents that can inactivate acrAB.

### EXAMPLE 10

### Isolation and Characterization of Mutants of M. smegmatis Selected for Resistance to Triclosan or to Isoniazid

Three Mycobacterium smegmatis mutants were selected for resistance to triclosan and were found to have different mutations in InhA, an enoyl reductase involved in fatty acid synthesis. Isoniazid resistance accompanied triclosan resistance for the Met161Val mutation and to a lesser extent for Ala124Val, but not for Met103Thr. A Ser94Ala mutation originally selected on isoniazid also mediated triclosan resistance, as did the wild type inhA eliminated resistance. These results suggest that M. smegmatis InhA, like its Escherichia coli homolog FabI, is a target for triclosan.

M. smegmatis strain mc²155 was grown in LB broth or 7H9 medium (see legend to Table 1) to stationary phase and approximately 10⁸ colony-forming units were plated onto LB agar (without Tween 80 or glycerol) containing 0.8-1.6 μgml⁻¹ triclosan (a trichlorinated diphenyl ether, from Ciba-Geigy Corp., Greensboro, N.C.). After a 3 day incubation, the largest of the 20-200 colonies of various sizes which appeared per plate were selected. Three independent mutants, MT1, MT9, and MT17, were chosen for study. Each was 4-6 times more resistant to triclosan than was the parental strain (Table 2). Mutant MT1 manifested considerable resistance to isoniazid, MT17 less, and MT9 none (Table 1). Mutant mc²651 (from W. R. Jacobs, Jr.), which has the S94A substitution in InhA (Banerjee, A. et al. 1994. Science 263:227-230), as expected showed isoniazid resistance. In addition, it had a 4-6 fold triclosan resistance (Table 2). The wild type M. smegmatis inhA gene on multicopy plasmid pMD31 ::inhA⁺ (an unpublished Kan^{R} E. coli-mycobacterial shuttle plasmid derived by subcloning a 3 kb BamHI fragment including orfl-inhA-orf3 into pMD31 (Donnelly-Wu et al. 1993. Mol. Microbiol. 7:407-417); gift of L. Miesel) caused resistance to triclosan and isoniazid (Table 1), likely related to target overexpression. These data suggested that M. smegmatis InhA is a target for triclosan.

### EXAMPLE 11

### Substitution of Wild Type inhA for Mutant inhA

If a mutation in inhA were responsible for both the triclosan and isoniazid resistance, homologous replacement of the mutant inhA chromosomal gene with a wild type inhA gene would eliminate the resistances. The method employed pYUB325 (Miesel, L. et al. 1998. J. Bacteriol. 180-2459-2467), from W. R. Jacobs, Jr.,), a shuttle cosmid containing a large PacI restriction fragment from the mc²155 genome. Within this fragment are the wild type inhA⁺ gene and a nearby kanamycin resistance gene insert. pYUB325 (prepared from E. coli host STBL-2 [Gibco/BRL]) was digested with PacI and extracted with phenol/chloroform. Cells in logarithmic phase in LB broth/0.2% Tween 80 were chilled on ice for 1.5hr and pelleted at 4° C. The pellets were resupended gently in 0.2 vol of cold 10% glycerol/ 0.1% Tween 80, and then 10% glycerol was added up to 1 vol. Cells were pelleted and the resuspension and washing process repeated once, with final resuspension in 0.01 vol of glycerol/Tween 80. Electroporation was performed using 0.1 ml cell suspension with 0.2 μgDNA in 0.2 cm chilled cuvettes at 2.5 kV, 25 μF, 1000Ω. Then 1 ml LB broth/0.5% Tween 80 was added, the cells grown fro 4-16hr, plated on LB agar containing 15 μg ml⁻¹ kanamycin, and incubated 4-6 days.

Four kanamycin resistant transformants of each mutant were assayed for drug susceptibility by agar dilution. All four transformants of mutant MT9, three of both MTI and mc²651, and one of MT17 had lost both triclosan and any isoniazid resistance. The rest retained the parental resistance phenotype. These results are compatible with the expected frequency of 30-70% for coinheritance of inhA⁺ and Kan^{R} (Miesel, L. et al. 1998. J. Bacteriol. 180:2459-2467). Therefore the mutant inhA gene, or a gene very closely linked to it, had been responsible for both resistances in each mutant.

### EXAMPLE 12

### DNA Sequence in inhA Gene from Mutants

The inhA gene in each of the three triclosan-selected mutants was sequenced. Chromosomal DNA was prepared as described (Ausubel, F. M. et al. 1996. Current Protocols in Molecular Biology, vol 1 John Wiley Sons, p. 2.4.1.) using a 2 hr preliminary incubation at 37° C. of cells with 4 mg ml⁻¹ lysozyme. Polymerase chain reaction (PCR) of the entire inhA gene was performed for each mutant using Taq DNA polymerase (Gibco/BRL) at 2 mM Mg⁺⁺ in EasyStart reaction tubes (Molecular Bio-Products). Primers LM026 (forward): 5′-AAAGCCCGGACACACAAGA-3′) (SEQ ID NO: 13) and LM027 (reverse): 5′-CGAACGACAGCAGTAGCAAG-3′ (SEQ ID NO:14) were chosen from sequences bracketing inhA (see GenBank accession number (173544) using the PRIME program of GCG and were annealed at 52° C. Both strands of the resulting 890 bp PCR product were sequenced (Tufts Core Facility) using the same two primers.

The inhA structural gene of each mutant differed by a single nucleotide from the wild type sequence (GenBank accession number U02530). Together with the other results, this finding proved that a mutated inhA gene was responsible for the triclosan resistance in each mutant. Mutant MT1 had replacement of methionine 161 (ATG) by valine (GTG), mutant MT9 had replacement of methionine 103 (ATG) by threonine (ACG), and mutant MT17 had replacement of alanine 124 (GCG) by valine (GTG).

### EXAMPLE 13

### InhA Mediates Triclosan Resistance in M. smegmatis

All three of the M. smegmatis InhA residues mutated in the present study, like those in FabI of triclosan-resistant E. coli (McMurry, L. M. et al. 1998. Nature 394:531-532), lie close to the NADH cofactor and putative acyl substrate binding sites (observed using the program STING (Neshich, G. R. et al. 1998. Submitted to Protein Data Bank Quarterly Newsletter 84.) with M. tuberculosis InhA [Protein Data Base 1ENY]. Triclosan might, like isoniazid (Rozwarski, D. A. 1998. Science 279:98-102) and diazaborine (Baldock, C. et al. 1996. Science 274:2107-2110), bind covalently to NADH. Resistance then might be explained, as for isoniazid (Basso, L. A. et al. 1998. J. Infect. Dis. 178:769-775) (Dessen, A. et al. 1995. Science 267:1638-1641) (Rozwarski, D. A. et al. 1998. Science 278:98-102), by reduced binding of NADH to the enzyme. In this regard, replacement of methionine 161, near the amino terminus of helix A5, by valine in M. smegmatis InhA leads to triclosan/isoniazid resistance. Replacement of the equivalent diazaborine-interacting (Baldock, C. et al. 1996. Science 274:2107-2110) methionine 159 of E. coli FabI by threonine led to triclosan, but not diazaborine, resistance (McMurry, L. M. et al. 1998. Nature 394:531-532). These substitutions may interfere with the hydrogen bond to NADH formed by the conserved lysine 165 one helical turn away (Baldock, C. et al. 1996. Science 274:2107-2110; Dessen, A. et al. 1995. Science 267:1638-1641; Rafferty, J. B. et al. 1995. Structure 3:927-938). Near methionine 161 in InhA is methionine 103, located in the loop connecting strand B4 to helix A4. Its replacement by threonine conferred only triclosan resistance. The third altered residue, alanine 134, is in the middle of helix A4, near but facing away from NADH. Since this residue seems to lie outside the putative active site, the resistance caused by substitution of a more bulky valine may occur by an indirect allosteric effect. Steric interference with binding of diazaborine to the putative fatty acyl substrate binding site of E. coli FabI has been suggested as the resistance mechanism for the G93S mutation (Baldock, C. et al. 1996. Science 274:2107-2110). Whether or not triclosan binds to NADH, this hydrophobic molecule might block fatty acyl substrate binding.

M. smegmatis is suseptible to triclosan whereas M. tuberculosis is not (Vischer, W. A. et al. 1974. Zbl. Bakt. Hyg., I. Abt. Orig. A 226:376-389). The four residues in M. smegmatis InhA which influence triclosan resitance, S94, M103, A124, and M161, are conserved in M. tuberculosis. They would not, therefore, identify any residues unique to M. tuberculosis InhA which might account for the intrinsic resistance. On the other hand, that resistance may be due to mechanisms unrelated to InhA, such as the activity of endogenous efflux pump(s) analogous to those which operate on triclosan in other organisms (McMurry, L. M. et al. FEMS Microbiol. Lett.; Schweizer, H. P. 1998. Antimicrob. Agents Chemother. 42:394-398).

TABLE 2
Characteristics of strains of Mycobacterium smegmatis.

| | | | relative MIC (S.D.) | | |
|---|---|---|---|---|---|
| | | Inha | triclosan | triclosan | isoniazid |
| Strain | Characteristics (reference) | mutation | (LB) | (7H9) | (7H9) |
| mc²155 | wild type (see Meisel et al | none | 1.0 | 1.0 | 1.0 |
| | 1998. J. Bacteriol. 180:2459) | | | | |
| MT1 | mc² 155 selected on triclosan | M161V | 4.9(0.9) | 6.3(2.0) | 8.5(2.5) |
| | (this work) | | | | |
| MT9 | mc² 155 selected on triclosan | M103T | 4.4(1.1) | 6.3(2.0) | 1.2(0.5) |
| | (this work) | | | | |
| MT17 | mc² 155 selected on triclosan | A124V | 4.0(1.2) | 5.8(1.7) | 2.0(0.7) |
| | (this work) | | | | |
| mc²651 | mc² 155 selected on isoniazid | S94A | 4.4(1.3) | 6.3(2.0) | 22(12) |
| | (See Banerjee et al. 1994. | | | | |
| | Science. 263:227). | | | | |
| mc²155/ | mc^{2 155 bearing multicopy} | none | 4.6(0.6) | 6.3(2.0) | >64 |
| pMD31::inhA⁺ | ^{inhA}³⁰ (see text) | | | | |

Table 2. Minimal inhibitory concerntrations (MICs) are expressed as ratios to the MIC of M. smegmatis mc²155. All MICs were determined on agar plates by 2-fold serial dilutions using logarithmic phase cells as described (McMurry, L. M. et al. 1998. Nature 394:531-532). Cells were grown with 0.05% Tween 80 either in LB broth or in 7H9 medium supplemented with ADC plus 0.2% glycerol and were tested on the corresponding solid media without Tween 80. All plates with triclosan also contained 0.1% ethanol. Less clumpong of cells during growth was seen in 7H9 than in LB, but the MIC for mutants in 7H9 agar approached the solubility limit of triclosan in this medium (50-100 μg ml⁻¹, observed visually). Results are means (+/−standard deviation [S.D.]) of 4-5 experiments. The MICs for mc²155 (in μg ml⁻¹) were: triclosan in LB, 0.61 (+/−0.15); triclosan in 7H9, 14 (+/−5); isoniazid in 7H9,7 (+/−2).

### EXAMPLE 14

### Overexpression of the Multidrug Efflux Pump Locus acrAB, or of mar A or soxS, Both Encoding Positive Regulators of acrAB, Decreased Susceptibility to Triclosan 2-Fold

Deletion of the acrAB locus increased the susceptibility to triclosan approximately 10-fold. Four of five clinical E. coli strains which overexpressed mar A or soxS also showed enhanced triclosan resistance. The acrAB locus was involved in the effects of triclosan upon both cell growth rate and cell lysis.

Triclosan inhibits the synthesis of lipids in Escherichia coli, presumably by action upon FabI, an enoyl reductase required for the synthesis of fatty acids (McMurry et al. (1998) Triclosan targets lipid synthesis. Nature 394, 531-532). At higher concentrations, triclosan also causes cell lysis (McMurry et al. (1998) Triclosan targets lipid synthesis. Nature 394, 531-532; Regos et al. (1974) Investigations on the mode of action of triclosan, a broad spectrum antimicrobial agent. Zbl. Bakt. Hyg., I. Abt. Orig. A 226, 390-401). AcrAB is a multidrug efflux pump in E. coli (Nikaido, H. (1996) Multidrug efflux pumps of Gram-negative bacteria. J. Bacteriol. 178, 5853-5859; Okusu et al. (1996) AcrAB efflux pump plays a major role in the antibiotic resistance phenotype of Esherichia coli multiple antibiotic-resistance(Mar) mutants. J. Bacteriol. 178, 306-308)whose normal physiological role is unknown, although it may assist in protection of cells against bile salts in the mammalian small intestine (Thanassi et al. (1997) Active efflux of bile salts by Escherichia coli. J. Bacteriol. 179, 2512-2518). AcrAB confers intrinsic resistance to many diverse, mostly lipophilic, compounds including antibiotics and disinfectants (Nikaido, H. (1996) Multidrug efflux pumps of Gram-negative bacteria. J. Bacteriol. 178, 5853-5859; Okusu et al. (1996) AcrAB efflux pump plays a major role in the antibiotic resistance phenotype of Esherichia coli multiple antibiotic-resistance(Mar) mutants. J. Bacteriol. 178, 306-308; Moken et al. (1997) Selection of multiple-antibiotic-resistant (Mar) mutants of Escherichia coli by using the disinfectant pine oil: roles of the mar and acrAB loci. Antimicrob. Chemother. 41, 2770-2772). The acrAB operon is upregulated by MarA (Ma et al. (1995) Genes acrA and acrB encode a stress-induced efflux system of Escherichia coli. Mol. Microbiol. 16, 45-55), a transcriptional activator encoded by the marRAB operon involved in multiple antibiotic resistance (Alekshun et al. (1997) Regulation of chromosomally mediated multiple antibiotic resistance: the mar regulation. Antimicrob. Agents Chemother. 41, 2067-2075). Mutations in the repressor gene marR lead to overexpression of marA (Alekshun et al. (1997) Regulation of chromosomally mediated multiple antibiotic resistance: the mar regulation. Antimicrob. Agents Chemother. 41, 2067-2075; Cohen et al. (1993) Genetic and functional analysis of the multiple antibiotic resistance (mar) locus in Escherichia coli. J. Bacteriol. 175, 1484-492); Seoane et al. (1995) Characterization of MarR, the repressor of the multiple antibiotic resistance (mar) operon of Escherichia coli. J. Bacteriol. 177, 3414-3419). The soxS gene encodes a MarA homolog (Alekshun et al. (1997) Regulation of chromosomally mediated multiple antibiotic resistance: the mar regulation. Antimicrob. Agents Chemother. 41, 2067-2075; Li et al. (1996) Sequence specificity for DNA binding by Escherichia coli SoxS and Rob proteins. Mol. Microbiol. 20, 937-945; Miller et al. (1996) Overlaps and parallels in the regulation of intrinsic multiple-antibiotic resistance in Escherichia coli. Mol. Microbiol. 21, 441-448) which also positively regulates acrAB (Ma et al. (1996) The local repressor AcrR plays a modulating role in the regulation of acrAB genes of Escherichia coli by global stress signals. Mol. Microbiol. 19, 101-112).

TABLE 3
Triclosan susceptibility of strains overexpressing marA
(mutations in marR), soxS (mutation in soxR),
or acrAB (mutation in acrR)

| | | Relative |
|---|---|---|
| | | MIC of |
| Strain (plasmid)/reference | Characteristics | triclosan^{a} |
| HH180 (Cohen et al. | Wild-type Δmar^{b} | 1.0 |
| (1993) Genetic and functional analysis | | |
| of the multiple antibiotic resistance | | |
| (mar) locus inEscherichia coli. J | | |
| Bacteriol. 175, 1484-492) | | |
| HH180 (pHHMI84) (Cohen et al. | Wild-type Δmar^{b} | 1.1 |
| (1993) Genetic and functional analysis | (mar+) | |
| of the multiple antibiotic resistance | | |
| (mar) locus in Escherichia coli. J. | | |
| Bacteriol. 175, 1484-492) | | |
| HH180(pHHM191) (Cohen et al. | Wild-type Δmar^{b} | 3.0 |
| (1993) Genetic and functional analysis | (marR2) | |
| of the multiple antibiotic resistance | | |
| (mar) locus in Escherichia coli. J. | | |
| Bacteriol. 175, 1484-492) | | |
| HH180(pHHM193) (Cohen et al. | Wild-type Δmar^{b} | 4.6 |
| (1993) Genetic and functional analysis | (marR5) | |
| of the multiple antibiotic resistance | | |
| (mar) locus in Escherichia coli. J | | |
| Bacteriol. 175, 1484-492) | | |
| GC4488 (Greenberg et al. (1991) | Wild-type | 1.0 |
| Activation of oxidative stress genes by | | |
| mutation at the soxQ1/cfxB1/marA | | |
| locus of Eseherichia coli. J. | | |
| Bacteriol. 173, 4433-4439) | | |
| JTG1078 (Greenberg et al. (1991) | GC4488 soxR105 | 2.1 |
| Activation of oxidative stress genes by | zjc-2204:: Tn 10kan | |
| mutation at the soxQ1/cfxB1/marA | | |
| locus of Eseherichia coli. J. | | |
| Bacteriol. 173, 4433-4439) | | |
| AG100 (George et al. (1983) | Wild-type | 1.0 |
| Amplifiable resistance to tetracycline, | | |
| chloramphenicol, and other antibiotics | | |
| in Escherichia coli: involvement of a | | |
| non-plasmid-determined efflux of | | |
| tetracycline. J. Bacteriol. 155, 531- | | |
| 540) | | |
| AG100B (Okusu et al. (1996) AcrAB | AG100 acrR:: kan | 1.9 |
| efflux pump plays a major role in the | | |
| antibiotic resistance phenotype of | | |
| Esherichia coli multiple antibiotic | | |
| resistance(Mar) mutants. J. Bacteriol | | |
| 178, 306-308) | | |

^{a}MIC of strain divided by MIC of corresponding wild-type strain. MIC for AG100 was 0.17 μg ml^{31 1}, for HH180, 0.07 μg ml⁻¹. and for GC4488, 0.08 μg ml⁻¹. MIC values are means from two to five determinations.
^{b}Has a 39 kb chromosomal deletion encompassing the mar locus.

Materials and Methods

All strains except those designated as ‘clinical’ were E. coli K-12 derivatives. Cells were grown in LB broth or on LB agar at 37° C. Minimal inhibitory concentration (MIC) was determined using serial dilution LB agar plates with steps of 1.2-1.5-fold increasing concentrations of triclosan (also called Irgasan DP300; a gift from Ciba-Geigy). A 5 μl amount of exponential phase cells at OD₅₃₀=0.01 (about 3×10⁴ colony-forming units) was applied to the agar and the MIC was defined as the lowest concentration which allowed no visible growth after 20 h at 37° C.

Results

Overexpression of the mar, sox, or acrAB Locus Decreased Susceptibility to Triclosan

Defined mutations in marR within the marRAB operon cloned on low copy plasmids (pHHM191, pHHM193) lead to overexpression of marA (Alekshun et al. (1997) Regulation of chromosomally mediated multiple antibiotic resistance: the mar regulation. Antimicrob. Agents Chemother. 41, 2067-2075; Cohen et al. (1993) Genetic and functional analysis of the multiple antibiotic resistance (mar) locus in Escherichia coli. J. Bacteriol. 175, 1484-492; Seoane et al. (1995) Characterization of MarR, the repressor of the multiple antibiotic resistance (mar) operon of Escherichia coli. J. Bacteriol. 177, 3414-3419). These mutations caused a 2.8-4.2-fold reduction in the susceptibility to triclosan as compared to the wild-type strain HH180/pHHM184 (deleted for the chromosomal mar locus and bearing the wild-type mar+ locus on a low copy plasmid) (Table 1). Chromosomal Mar mutants (overexpressing marA) showed a 2-fold lower susceptibility to triclosan (Table 2, strains AG102 and AP5). Strain JTG1078, overexpressing soxS, had a triclosan MIC twice that of its parental strain GC4488 (Table 1). Overexpression of acrAB resulting from a mutation in acrR doubled the triclosan MIC (strain AG100B, Table 3).

Effect of Deletion of the mar or acrAB Locus on Susceptibility to Triclosan

Deletion of the marCORAB locus from wild-type strain AG100had little effect on susceptibility to triclosan, while deletion from Mar mutants AG102 and AP5 eliminated their resistance to triclosan (Table 4). Deletion of the acrAB locus increased the triclosan susceptibility about 10-fold in parental strain AG100 and some 20-fold in the Mar mutants AG102 and AP5, thereby equalizing the susceptibility of the two classes of strains (Table 4). Evidently, the amount of MarA in a Mar mutant, but not in the wild type strain, was sufficient to up-regulate acrAB.

TABLE 4
Effect of deletion of the marCORAB acrAB locus upon susceptibility to triclosan

| | | Relative MIC of triclosan^{a} | | |
|---|---|---|---|---|
| Parental strain/reference | Characteristics | Control | ΔmarCORAB^{b} | ΔacrAB^{b} |
| AG100 (George et al. (1983) Amplifiable | Wild-type | 1.0 | 0.87 | 0.11 |
| resistance to tetracycline, | | | | |
| chloramphenicol, and other antibiotics in | | | | |
| in Escherichia coli: involvement of a | | | | |
| non-plasmid-determined efflux of | | | | |
| tetracycline. | | | | |
| J. Bacterial. 155, 531-540) | | | | |
| AG102 (Cohen et al. (1993) Genetic and | AG100 marR1 | 2.0 | 0.86 | 0.092 |
| functional analysis of the multiple | | | | |
| antibiotic resistance (mar) locus in | | | | |
| Escherichia coli. J. Bacteriol. | | | | |
| 175, 1484-492) | | | | |
| AP5 (Nikaido, H. (1996) Multi-drug | AG100 mar | 2.0 | 0.95 | 0.092 |
| efflux pumps of Gram-negative bacteria. | | | | |
| J. Bacteriol. 178, 5853-5859) | | | | |

Strains AG102 and AP5 are chromosomal Mar mutants and overexpress marA.
^{a}MIC of strain divided by MIC of AG100 control strain (with no deletion). The MIC for AG100 was 0.17 μg ml⁻¹
^{b}Parental strain with this additional deletion; construction of these inactivated strains has been described (Moken et al. (1997) Selection of multiple-antibiotic-resistant (Mar) mutants of Escherichia coli by using the disinfectant pine oil: roles of the mar and acrAB loci. Antimicrob. Chemother. 41, 2770-2772).

Deletion of acrAB Decreased the Concentration of Triclosan Required for Cell Lysis

Use of liquid cultures permitted both growth rate and cell lysis to be monitored. Lysis was defined by loss of absorbance together with loss of viability. AG100 in liquid culture required 0.6 μg ml⁻¹ triclosan to inhibit the growth rate 90% but 8 μg ml⁻¹ for lysis (Table 5). Since the MIC (determined on agar) was 0.17-0.28 μg ml⁻¹ for AG100 (Tables 3-5), the MIC values almost surely reflected growth inhibition rather than cell lysis. That deletion of the acrAB locus decreased the MIC for triclosan 10-fold (Table 4) suggested that the AcrAB efflux pump lowers the internal concentration of triclosan affecting enoyl reductase, a cytoplasmic enzyme which is the putative target of triclosan (McMurry et al. (1998) Triclosan targets lipid synthesis. Nature 394, 531-532) and which is essential for cell growth (Cronan et al. (1996) Biosynthesis of membrane lipids. In: Escherichia coli and Salmonella: Cellular and Molecular Biology (Neidhardt, F. C., Ed.), pp. 612-636, ASM Press, Washington, D.C.).

AcrAB also influenced the effect of triclosan upon cell lysis. The susceptibility of wild-type cells to lysis by triclosan was increased about 2-fold by loss of the efflux pump (Table 5). The mechanism of triclosan-induced lysis is not known. However, the G93V mutation in enoyl reductase in triclosan-resistant mutant AGT11 (isogenic with AG100; (McMurry et al. (1998) Triclosan targets lipid synthesis. Nature 394,531-532)) led to resistance of cells both to growth rate inhibition and to lysis (Table 5; (McMurry et al. (1998) Triclosan targets lipid synthesis. Nature 394, 531-532)), suggesting that synthesis of fatty acids/lipids might not only be needed for growth but also to prevent lysis. On the other hand, when the acrAB locus was deleted from AGT11, notable protection by the G93V mutation remained against growth rate inhibition but not against lysis (Table 5, AG100A vs. AGT11K). If the AcrAB pump were to remove drugs, such as triclosan, directly from the membrane (Nikaido, H. (1996) Multidrug efflux pumps of Gram-negative bacteria. J. Bacteriol. 178, 5853-5859), loss of this pump might allow the hydrophobic triclosan to accumulate in the membrane bilayer to a critical level leading to lysis regardless of the rate of fatty acid synthesis.

TABLE 5
Concentration of triclosan required in liquid culture to inhibit growth and to cause lysis in strains deleted
for acrAB and/or bearing afabl mutation mediating triclosan resistance

| | | | Concentration (μg ml-1) of triclosan which | |
|---|---|---|---|---|
| Strain/reference | Characteristics | MIC^{a} (μg ml⁻¹) | inhibited growth rate 50% (90%) | Caused lysis |
| AG100 (George et al. | Wild-type | 0.28 | 0.15 (0.6) | 8 |
| (1983) Amplifiable | | | | |
| resistance to tetracycline, | | | | |
| chloramphenicol, and other | | | | |
| antibiotics in Escherichia | | | | |
| coli: involvement of a non- | | | | |
| plasmid-determined efflux | | | | |
| of tetracycline. J. | | | | |
| Bacteriol. 155, 531-540) | | | | |
| AG100A (Okuso et al. | AG100 ΔacrAB::kan | 0.018 | 0.02 (0.05) | 3-4 |
| (1996) AcrAB efflux pump | | | | |
| plays a major role in the | | | | |
| antibiotic resistance | | | | |
| phenotype of Esherichia | | | | |
| coli, multiple antibiotic- | | | | |
| resistance(Mar) mutants. J | | | | |
| Bacteriol. 178. 306-308) | | | | |
| AGT11 (McMuny et al. | AG100fabI(G93V) | 41 | 13 (>32)^{b} | >32^{b} |
| (1998) Triclosan targets | | | | |
| lipid synthesis. Nature | | | | |
| 394, 531-532) | | | | |
| AGT11K^{c} (this work) | AGT11 ΔacrAB::kan | 3.2 | 1.3 (2.1) | 3-4 |
| The concentration of triclosan required to slow the growth rate by 50% (or 90%) an hour after addition was determined using OD₅₃₀ to monitor growth. ‘Lysis’ was defined in such cultures by a 30-50% loss of OD₅₃₀ within 2 h of triclosan addition accompanied by a 4-6 log loss in viability (as indicated by colony-forming units). | | | | |
| ^{a}Determined using agar dilution plates. | | | | |
| ^{b}Triclosan formed an insoluble precipitate above 32 μg ml⁻¹; no lysis of cells was seen even at nominal triclosan concentrations of 256 μg ml⁻. | | | | |
| ^{c}AFT11K was constructed by P1 transduction (Provence et al. (1994) Gene transfer in Gram-negative bacteria. In: Methods for General and Molecular Bacteriology (Gerhardt et al.), pp. 317-347. American Society for Microbiology, Washington, D.C.) of ΔacrAB::kan from strain JZM120 (Okusu et al. (1996) AcrAB efflux pump plays a major role in the antibiotic resistance phenotype of Esherichia coli multiple antibiotic-resistance(Mar) mutants. J. Bacteriol. 178, 306-308; | | | | |
| # Ma et al. (1995) Genes acrA and acrB encode a stress-induced efflux system of Escherichia coli. Mol. Microbial. 16, 45-55) into AGT11. | | | | |

Relationship of Triclosan Susceptibility to Overexpression of marA or soxS in Clinical Strains

Triclosan susceptibility of clinical strains of E. coli from blood samples taken in hematology-oncology hospital wards in Europe (Oethinger et al. (1997) Association of organic solvent tolerance and fluoroquinolone resistance in clinical isolates of Escherichia coli. J. Antimicrob. Chemother. 41, 111-114). All strains chosen from Series S were susceptible to fluoroquinolones, tetracycline, ampicillin, and chloramphenicol, while all chosen from series HO and E were resistant to all four antibiotics. Of 15 susceptible strains, 14 had a mean triclosan MIC of 0.090 μg ml⁻¹ (S.D. 0.014). The remaining susceptible strain, S20, was exceptional in overexpressing marA (Oethinger et al. (1998) Overexpression of te regulatory marA or soxS gene in clinicial topoisomerase mutants of Escherichia coli. Antimicrob. Agents Chemother. 42, 2089-2094) and had a correspondingly higher triclosan MIC, 0.27 μg ml⁻¹. Of 31 multiply resistant strains, three (E3, E19, HO99) overexpressed either marA or soxS (Oethinger et al. (1998) Overexpression of te regulatory marA or soxS gene in clinicial topoisomerase mutants of Escherichia coli. Antimicrob. Agents Chemother. 42, 2089-2094), which correlated with a higher mean triclosan MIC of 0.33 μg ml⁻¹ (S.D. 0.03); the fourth strain (HO 17) also overexpressed marA (Oethinger et al. (1998) Overexpression of te regulatory marA or soxS gene in clinicial topoisomerase mutants of Escherichia coli. Antimicrob. Agents Chemother. 42, 2089-2094) but had a triclosan MIC of only 0.15 μg ml⁻¹. Multiply resistant strain E10 had a triclosan MIC of 0.38 μg ml⁻¹, but overexpressed neither marA or soxS, nor was it tolerant to cyclohexane (Oethinger et al. (1998) Overexpression of tHe regulatory marA or soxS gene in clinicial topoisomerase mutants of Escherichia coli. Antimicrob. Agents Chemother. 42, 2089-2094), a hallmark of strains overexpressing marA, soxS, robA, or acrAB (White et al. (1997) Role of the acrAB locus in organic solvent tolerance mediated by expression of marA, soxS, or robA in Escherichia coli. J. Bacteriol. 179, 6122-6126). This strain probably has mutations(s) at other loci, possibly including fabI. The remaining 26 multiply resistant strains overexpressing neither marA or soxS had a mean triclosan MIC of 0.13 μg ml⁻¹ (S.D. 0.04). In summary, regardless of the multiple antibiotic resistance phenotypes, four of the five clinical strains which overexpressed marA or soxS had a triclosan MIC more than twice that of strains which did not overexpress either gene. This effect was consistent with the findings in the laboratory K-12 strains.

Discussion

The deletion of AcrAB multidrug efflux pump increases the susceptibility of E. coli strains to triclosan, both at the level of growth inhibition and of lysis. Triclosan can now be added to the list (Nikaido, H. (1996) Multidrug efflux pumps of Gram-negative bacteria. J. Bacteriol. 178, 5853-5859) of presumed AcrAB substrates. In Pseudomonas aeruginosa, a recent study indicates that triclosan is also a substrate for the MexAB-OprM multidrug efflux pump (Schweizer, H. P. (1998) Intrinsic resistance to inhibitors of fatty acid biosynthesis in Psuedomonas aeruginosa is due to efflux: application of a novel technique for generation of unmarked chromosomal mutations for the study of efflux systems. Antimicrob. Agents Chemother. 42, 394-398). Mutations at the secondary loci acr, mar, and sox in E. coli conferred only a 2-fold resistance to triclosan, presumably via a small up-regulation of acrAB. A mutation at any one of these three loci might not by itself threaten triclosan efficacy, but might act synergistically with mutations at other loci such as fabI, where mutations can increase triclosan resistance 90-140-fold (Table 5). Finally, low levels of the very stable triclosan in the environment might encourage preferential survival of acrlmarlsox mutants resistant to multiple antibiotics.

The contents of all references, pending patent applications and published patents, cited throughout this application are hereby expressly incorporated by reference. In addition, the contents of McMurry et al. 1998. FEMS Microbiology Letters 166:305 are also expressly incorporated by this reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for identifying an antimicrobial compound which interacts with an enoyl ACP reductase (ER) polypeptide, comprising;
contacting the ER polypeptide with a compound under conditions which allow interaction of the compound with the ER polypeptide to occur; and
detecting the presence or absence of interaction of the compound with the ER polypeptide as an indication of whether the compound is an antimicrobial compound.

2. The method of claim 1 wherein the ER polypeptide is selected from the group consisting of a FabI polypeptide and an InhA polypeptide.

3. The method of claim 1 wherein the compound in the contacting step is a compound categorized as an NSAM.

4. The method of claim 1 wherein the compound is a triclosan compound.

5. The method of claim 1 wherein the compound is not selected from the group consisting of isoniazid, diazaborine, and ethionamide.

6. The method of claim 1 wherein the compound is not an antibiotic.

7. The method of claim 1 or 2 wherein the interaction occurs with the NAD/NADP binding cleft of the ER polypeptide.

8. The method of claim 1 or 2 wherein the interaction occurs within the triclosan binding portion of the ER polypeptide.

9. The method of claim 1 wherein the interaction is detected based on the presence or absence of enzyme activity.

10. A method for identifying an antimicrobial compound, comprising;
contacting an enoyl reductase molecule with a compound under conditions which allows enzyme activity to occur; and
detecting the presence or absence of enzyme activity as an indication of whether the compound is an antimicrobial compound.

11. The method of claim 10 wherein the antimicrobial is an antibacterial.

12. The method of claim 10 wherein the compound in the contacting step is a compound categorized as an NSAM.

13. The method of claim 10 wherein the compound is a triclosan compound.

14. The method of claim 10 wherein the compound is not selected from the group consisting of isoniazid, diazaborine, and ethionamide.

15. The method of claim 10 wherein the compound is not an antibiotic.

16. The method of claim 10 wherein the ER polypeptide is selected from the group consisting of a FabI polypeptide and an InhA polypeptide.

17. A method for identifying an antimicrobial compound, comprising;
exposing a microorganism to a compound under conditions which allow fatty acid biosynthesis to occur; and
detecting the inhibition of fatty biosynthesis as an indication of whether the compound is an antimicrobial compound.

18. The method of claim 17 wherein the antimicrobial is an antibacterial.

19. The method of claim 17 wherein the compound in the exposing step is a compound categorized as an NSAM.

20. The method of claim 17 wherein the compound is a triclosan compound.

21. The method of claim 17 wherein the compound is not selected from the group consisting of isoniazid, diazaborine, and ethionamide.

22. The method of claim 17 wherein the compound is not an antibiotic.

23. The method of claim 17 wherein the ER polypeptide is selected from the group consisting of a FabI polypeptide and an InhA polypeptide.

24. A method for identifying an antimicrobial compound which interacts with a mutant ER polypeptide, comprising;
contacting the mutant ER polypeptide with a compound under conditions which allow interaction of the compound to the mutant ER polypeptide to occur; and
detecting the presence or absence of interaction with the mutant ER polypeptide as an indication of whether the compound is an antimicrobial compound.

25. The method of claim 24 wherein the mutant ER polypeptide does not interact with triclosan and the compounds being contacted are triclosan compounds.

26. The method of claim 24 wherein the ER is selected from the group consisting of a FabI polypeptide and an InhA polypeptide..

27. The method of claim 24 wherein the compound in the contacting step is a compound categorized as an NSAM.

28. The method of claim 24 wherein the compound is a triclosan compound.

29. The method of claim 24 wherein the binding occurs within the reducing agent binding cleft of the mutant FabI polypeptide.

30. The method of claim 24 wherein the binding occurs within the triclosan binding cleft of the mutant FabI polypeptide.

30. The method of claim 24 wherein the binding occurs within the NAD/NADP binding cleft of the mutant FabI polypeptide.

31. The method of claim 24 wherein the binding is detected based on the presence or absence of enzyme activity.

32. The method of claim 24 wherein the mutant ER polypeptide has an altered amino acid in the NAD/NADP binding cleft.

33. The method of claim 24 wherein the ER polypeptide is selected from the group consisting of a FabI polypeptide and an InhA polypeptide.

34. The method of claim 24 wherein the ER polypeptide is a mutant FabI polypeptide having an altered amino acid at residue 93.

35. The method of claim 24 wherein the ER polypeptide is a mutant FabI polypeptide havingmutant FabI polypeptide has an altered amino acid at residue 159 or 203.

36. The method of claim 24 wherein the ER polypeptide is a mutant FabI polypeptide having a gly93val substitution.

37. The method of claim 24 wherein the ER polypeptide is a mutant FabI polypeptide having a substitution selected from the group consisting of met159thr and phe203leu.

38. A method for identifying an antimicrobial compound capable of inhibiting proliferation or viability of a triclosan-resistant microbial cell, comprising
contacting a triclosan-resistant microbial cell with a compound under conditions which allow a triclosan-resistant microbial cell to proliferate or remain viable;
determining whether the compound is capable of inhibiting proliferation or viability of the cell thereby identifying an antimicrobial compound capable of inhibiting proliferation or viability of a triclosan-resistant microbial cell.

39. The method of claim 38, wherein lysis of the triclosan-resistant microbial cell is used in the determining step to identify an antimicrobial compound capable of inhibiting proliferation or viability of a triclosan-resistant cell.

40. The method of claim 38, wherein the triclosan-resistant microbial cell comprises a mutant FabI polypeptide having the substitution gly93val.

41. The method of claim 38, wherein the triclosan-resistant microbial cell comprises a mutant FabI polypeptide having a mutation selected from the group consisting of met159thr and phe203leu39.

42. The method of claim 38, wherein the triclosan-resistant microbial cell is acrAB⁺.

43. The method of claim 38, wherein the cell is AGT11.

44. The method of claim 38, wherein the cell is AGT23.

45. The method of claim 38, wherein the cell is AGT25.

46. A method for identifying an antimicrobial compound capable of inhibiting proliferation or viability of a triclosan-resistant microbial cell, comprising;
contacting a polypeptide capable of conferring resistance to triclosan with a compound under conditions which allow interaction of the compound to the polypeptide to occur; and
detecting the presence or absence of interaction with the polypeptide as an indication of whether the compound is an antimicrobial compound capable of inhibiting proliferation or viability of a triclosan-resistant microbial cell.

47. The method of claim 46 wherein the compound is a triclosan compound.

48. A method for identifying an antimicrobial compound capable of inhibiting proliferation or viability of a NSAM-resistant microbial cell, comprising;
contacting a polypeptide capable of conferring resistance to a NSAM with a compound under conditions which allows interaction of the compound with the polypeptide to occur; and
detecting the presence or absence of interaction with the polypeptide as an indication of whether the compound is an antimicrobial compound capable of inhibiting proliferation or viability of a NSAM-resistant microbial cell.

49. The method of claim 48 wherein the compound is a NSAM compound which is a structural analog of the parent NSAM compound.

50. A method for identifying an antimicrobial compound capable of inhibiting proliferation or viability of a NSAM-resistant microbial cell, comprising
contacting a a NSAM-resistant microbial cell with a compound under conditions which allow a a NSAM-resistant microbial cell to proliferate or remain viable;
determining whether the compound is capable of inhibiting proliferation or viability of the cell thereby identifying an antimicrobial compound capable of inhibiting proliferation or viability of a a NSAM-resistant microbial cell.

51. An antimicrobial compound identified using any one of the methods of claims 1, 24, and 38.

52. A combination product comprising a compound of claim 50 and a product forming a combination product.

53. The combination product of claim 52 wherein the product is selected from the group consisting of detergent, soap, deodorant, disinfectant, mouthwash and toothpaste.

54. A combination product comprising a structural analog of triclosan and a product forming a combination product.

55. The combination product of claim 54 wherein the product is selected from the group consisting of detergent, soap, deodorant, disinfectant, mouthwash and toothpaste.

56. A combination product comprising a structural analog of an NSAM and a product forming a combination product.

57. The combination product of claim 56 wherein the product is selected from the group consisting of detergent, soap, deodorant, disinfectant, mouthwash and toothpaste.

58. The methods of any one of claims 1, 24, and 38 wherein the antimicrobial agent is antimicrobial for a microbial cell selected from the group consisting of a gram negative bacterium, a gram positive bacterium, a fungus, a spirochete, and a protozoan.

59. The method of claim 58, wherein the microbial cell is a gram negative bacterium.

60. The method of claim 59, wherein the gram negative bacterium is selected from the group consisting of Escherichia, Campylobacter, Salmonella, Shigella, Klebsiella, Helicobacter, Erwinia, Serratia, Yersinia, and Pseudomonas.

61. The method of claim 58, wherein the microbial cell is a gram positive bacterium.

62. The method of claim 61, wherein the gram positive bacterium is selected from the group consisting of is selected from the group consisting of Streptococcus, Listeria, Actinomyces, Mycobacterium, Sarcina, Staphylococcus, and Enterococcus.

63. The method of claim 58, wherein the microbial cell is a fungus.

64. The method of claim 63, wherein the fungus is Candida.

65. The method of claim 58, wherein the microbial cell is a protozoan.

66. The method of claim 58, wherein the microbial cell is a spirochete.

67. The method of claim 66, wherein the spirochete is selected from the group consisting of a Borrelia, a Leptonema, a Leptospira, a Spirochaeta, and a Treponema.

68. An isolated polypeptide capable of conferring resistance to a NSAM in a microbial cell.

69. An isolated polypeptide capable of conferring resistance to triclosan in a microbial cell.

70. The isolated polypeptide of claim 68 or 69, wherein the polypeptide is capable of conferring resistance to a bacterial cell.

71. The isolated polypeptide of claim 68 or 69, wherein the resistance is ability of the resistant mutant to grow in the presence of greater than four-fold the minimum inhibitory concentration of the microbial cell in the absence of the mutant polypeptide.

72. An isolated mutant ER polypeptide capable of conferring resistance to triclosan in a microbial cell.

73. The isolated mutant ER of claim 72, wherein the ER is selected from the group consisting of a FabI polypeptide and an InhA polypeptide.

74. The isolated mutant ER polypeptide of claim 73, wherein the ER is a FabI polypeptide having a gly93val substitution.

75. The isolated mutant ER polypeptide of claim 73 wherein the ER is a FabI polypeptide having a substitution selected from the group consisting of met159thr and phe203leu.

76. The isolated mutant ER polypeptide of claim 73 wherein the ER is a FabI polypeptide having an alteration of at least one amino acid in the NAD/NADP binding cleft.

77. The isolated mutant ER polypeptide of claim 73, wherein the mutant FabI polypeptide is a FabI polypeptide having has an amino acid sequence as shown in SEQ ID NO: 3 except for a mutation selected from the group consisting of G13, S16, S19, I20, A21, S91, I92, G93, F94, A95, L100, L144, S145, Y156, M159, K163, G190, P191, I192, R193, T194, L195, A196, I200, K201, D202, F203, R204 and K205.

78. An isolated nucleic acid encoding a mutant polypeptide as claimed in any one of claims 68, 69, 72 and 76.

79. An isolated microbial cell having a mutant polypeptide as claimed in any one of claims 68, 69, 72 and 76.

80. A method for treating a subject having growth of an unwanted microorganism with a NSAM, comprising:
administering to the subject an effective amount of the NSAM such that the subject is treated for the unwanted microorganism.

81. A method for treating a subject having growth of an unwanted microorganism with a triclosan compound, comprising:
administering to the subject an effective amount of the triclosan compound such that the subject is treated for the unwanted microorganism.

82. An antibody which specifically binds a mutant polvpeptide as claimed in any one of claims 68, 69, 72 and 76.

83. The antibody of claim 81 wherein the antibody does not bind a wild-type ER polypeptide.

84. The antibody of claim 83 which is a monoclonal antibody.

85. An antimicrobial soap or detergent preparation comprising triclosan at a concentration of less than about 500 μg per milliliter of soap or detergent preparation forming an antimicrobial soap or detergent preparation.

86. The antimicrobial soap or detergent preparation of claim 85 wherein triclosan is at a concentration of less than about 100 μg ml⁻¹.

87. The antimicrobial soap or detergent preparation of claim 85 wherein triclosan is at a concentration of less than about 50 μg ml⁻¹.

88. The antimicrobial soap or detergent preparation of claim 85 wherein triclosan is at a concentration of less than about 10 μg ml⁻¹.

89. The antimicrobial soap or detergent preparation of claim 85 wherein triclosan is at a concentration of less than about 10 μg ml⁻¹.

90. An antimicrobial soap or detergent preparation comprising a structural analog of triclosan in a soap or detergent preparation forming an antimicrobial soap or detergent preparation, said structural analog of triclosan capable of inhibiting the proliferation and viability of a triclosan-resistant microbial cell.

91. A method for screening a library of bacteriophage displaying on their surface a plurality of polypeptide sequences, each said polypeptide sequence being encoded by a nucleic acid contained within the bacteriophage, for ability to bind an immobilized ER fatty acid enoyl reductase molecule, to obtain those polypeptides having affinity for the enoyl reductase, said method comprising
contacting the immobilized enoyl reductase with a sample of the library of bacteriophage so that the enoyl reductase can interact with the different polypeptide sequences and bind those having affinity for the enoyl reductase to form a set of complexes consisting of immobilized enoyl reductase and bound bacteriophage;
separating the complexes from free bacteriophage which have not formed the complex;
contacting the complexes of the enoyl reductase and bound bacteriophage with an agent that dissociates the bound bacteriophage from the complexes; and
isolating the dissociated bacteriophage and obtaining the sequence of the nucleic acid encoding the displayed polypeptide, so that amino acid sequences of displayed polypeptides with affinity for fatty acid enoyl reductase are obtained.

92. The method of claim 17 wherein the microorganism is exposed to the compound in the presence of an inhibitor of an efflux pump.

93. The method of claim 92, wherein the efflux pump is AcrAB.

94. The method of claim 38 wherein the triclosan-resistant microbial cell is contacted with the compound in the presence of an inhibitor of an efflux pump.

95. The method of claim 94, wherein the efflux pump is AcrAB.

96. The antimicrobial compound of claim 51, wherein the minimum inhibitory concentration (MIC) of the compound is decreased in the presence of an inhibitor of the AcrAB efflux pump.

97. The antimicrobial compound of claim 96, wherein the decrease in MIC in the presence of the inhibitor of the AcrAB efflux pump is at least four-fold.

98. The antimicrobial compound of claim 97, wherein the decrease in MIC in the presence of the inhibitor of the AcrAB efflux pump is at least ten-fold.

99. The method of claim 80, wherein the subject is additionally treated with an efflux pump inhibitor.


SEQUENCE LISTING (14 sequences)

SEQ ID NO: 1 - DNA, 1301 bp, Escherichia coli
  CDS (14)..(232)
  CDS (236)..(343)
  CDS (347)..(1189)
  CDS (1193)..(1204)
  CDS (1208)..(1300)
ctgcaggaactgaaccgccggtcaccctctccctgaaagagcgagggggcagaccgagccgaatagctgttgtggtgaaaacatggagacggtgctggagaatattcggcaaggtctgaaccgtcccagccatcgccatgaaagggttaggggctgtatgagcctgtttgttgctggggtaacaatatttgcacaatacggtcccctcgcccctctggggagagggttagggtgaggggaaaagcgccccccctgccgcagcctgctccggtcggacctggcaactatagctactcacagccaggttgattataataaccgtttatctgttcgtactgtttactaaaacgacgaatcgcctgattttcaggcacaacaagcatcaacaataaggattaaagctatgggttttctttccggtaagcgcattctggtaaccggtgttgccagcaaactatccatcgcctacggtatcgctcaggcgatgcaccgcgaaggagctgaactggcattcacctaccagaacgacaaactgaaaggccgcgtagaagaatttgccgctcaattgggttctgacatcgttctgcagtgcgatgttgcagaagatgccagcatcgacaccatgttcgctgaactggggaaagtttggccgaaatttgacggtttcgtacactctattggttttgcacctggcgatcagctggatggtgactatgttaacgccgttacccgtgaaggcttcaaaattgcccacgacatcagctcctacagcttcgttgcaatggcaaaagcttgccgctccatgctgaatccgggttctgccctgctgaccctttcctaccttggcgctgagcgcgctatcccgaactacaacgttatgggtctggcaaaagcgtctctggaagcgaacgtgcgctatatggcgaacgcgatgggtccggaaggtgtgcgtgttaacgccatctctgctggtccgatccgtactctggcggcctccggtatcaaagacttccgcaaaatgctggctcattgcgaagccgttaccccgattcgccgtaccgttactattgaagatgtgggtaactctgcggcattcctgtgctccgatctctctgccggtatctccggtgaagtggtccacgttgacggcggtttcagcattgctgcaatgaacgaactcgaactgaaataatcgttctgttggtaaagatgggcggcgttctgccgcccgttatctctgttatacctttctgatatttgttatcgccgatccgtctttctccccttcccgccttgcgtca

SEQ ID NO: 2 - DNA, 786 bp, Escherichia coli
  CDS (1)..(786)
atgggttttctttccggtaagcgcattctggtaaccggtgttgccagcaaactatccatcgcctacggtatcgctcaggcgatgcaccgcgaaggagctgaactggcattcacctaccagaacgacaaactgaaaggccgcgtagaagaatttgccgctcaattgggttctgacatcgttctgcagtgcgatgttgcagaagatgccagcatcgacaccatgttcgctgaactggggaaagtttggccgaaatttgacggtttcgtacactctattggttttgcacctggcgatcagctggatggtgactatgttaacgccgttacccgtgaaggcttcaaaattgcccacgacatcagctcctacagcttcgttgcaatggcaaaagcttgccgctccatgctgaatccgggttctgccctgctgaccctttcctaccttggcgctgagcgcgctatcccgaactacaacgttatgggtctggcaaaagcgtctctggaagcgaacgtgcgctatatggcgaacgcgatgggtccggaaggtgtgcgtgttaacgccatctctgctggtccgatccgtactctggcggcctccggtatcaaagacttccgcaaaatgctggctcattgcgaagccgttaccccgattcgccgtaccgttactattgaagatgtgggtaactctgcggcattcctgtgctccgatctctctgccggtatctccggtgaagtggtccacgttgacggcggtttcagcattgctgcaatgaacgaactcgaactgaaa

SEQ ID NO: 3 - protein, 262 aa, Escherichia coli
MGFLSGKRILVTGVASKLSIAYGIAQAMHREGAELAFTYQNDKLKGRVEEFAAQLGSDIVLQCDVAEDASIDTMFAELGKVWPKFDGFVHSIGFAPGDQLDGDYVNAVTREGFKIAHDISSYSFVAMAKACRSMLNPGSALLTLSYLGAERAIPNYNVMGLAKASLEANVRYMANAMGPEGVRVNAISAGPIRTLAASGIKDFRKMLAHCEAVTPIRRTVTIEDVGNSAAFLCSDLSAGISGEVVHVDGGFSIAAMNELELK

SEQ ID NO: 4 - DNA, 20 bp, synthetic construct
gagcctgtttgttgctgggg

SEQ ID NO: 5 - DNA, 20 bp, synthetic construct
tgcagcaatgctgaaaccgc

SEQ ID NO: 6 - DNA, 17 bp, synthetic construct
cgggaaggggagaaaga

SEQ ID NO: 7 - DNA, 18 bp, synthetic construct
aattgcccacgacatcag

SEQ ID NO: 8 - DNA, 20 bp, synthetic construct
cgttgtagttcgggatagcg

SEQ ID NO: 9 - DNA, 16 bp, synthetic construct
cgcgatcatggcgacc

SEQ ID NO: 10 - DNA, 19 bp, synthetic construct
accagcgtttctgggtgag

SEQ ID NO: 11 - DNA, 850 bp, Mycobacterium smegmatis
  CDS (40)..(849)
ccggacacacaagatttctcgctcacaaggagtcaccaaatgacaggcctactcgaaggcaagcgcatcctcgtcacggggatcatcaccgattcgtcgatcgcgttccacatcgccaaggtcgcccaggaggccggcgccgaactggtgctgaccggtttcgaccgcctgaagttggtcaagcgcatcgccgaccgcctgcccaagccggccccgctgctggaactcgacgtgcagaacgaggagcacctgtcgactctggccgaccggatcaccgccgagatcggtgagggcaacaagatcgacggtgtggtgcactcgatcgggttcatgccgcagagcggtatgggcatcaacccgttcttcgacgcgccgtacgaggatgtgtccaagggcatccacatctcggcgtactcgtacgcctcgctcgccaaagccgttctgccgatcatgaatccgggcggcggcatcgtcggcatggacttcgaccccacgcgcgcgatgccggcctacaactggatgaccgtcgccaagagcgcgctcgaatcggtcaaccggttcgtcgcgcgtgaggcgggcaaggtgggcgtgcgctcgaatctcgttgcggcaggaccgatccgcacgctggcgatgagcgcaatcgtgggcggtgcgctgggcgacgaggccggccagcagatgcagctgctcgaagagggctgggatcagcgcgcgccgctgggctggaacatgaaggacccgacgcccgtcgccaagaccgtgtgcgcactgctgtcggactggctgccggccaccaccggcaccgtgatctacgccgacggcggcgccagcacgcagctgttgtgat

SEQ ID NO: 12 - protein, 269 aa, Mycobacterium smegmatis
MTGLLEGKRILVTGIITDSSIAFHIAKVAQEAGAELVLTGFDRLKLVKRIADRLPKPAPLLELDVQNEEHLSTLADRITAEIGEGNKIDGVVHSIGFMPQSGMGINPFFDAPYEDVSKGIHISAYSYASLAKAVLPIMNPGGGIVGMDFDPTRAMPAYNWMTVAKSALESVNRFVAREAGKVGVRSNLVAAGPIRTLAMSAIVGGALGDEAGQQMQLLEEGWDQRAPLGWNMKDPTPVAKTVCALLSDWLPATTGTVIYADGGASTQLL

SEQ ID NO: 13 - DNA, 19 bp, synthetic construct
aaagcccggacacacaaga

SEQ ID NO: 14 - DNA, 20 bp, synthetic construct
cgaacgacagcagtagcaag